(19) United States
(12) Patent Application Publication
(10) Pub. No.: US 2024/0317762 A1
(43) Pub. Date: Sep. 26, 2024

(54) PHTHALAZINONE-BASED PARP-1 INHIBITORS

(71) Applicant: Oregon Health & Science University, Portland, OR (US)

(72) Inventors: Michael Cohen, Portland, OR (US); Moriah Arnold, Portland, OR (US)

(73) Assignee: Oregon Health & Science University, Portland, OR (US)

(21) Appl. No.: 18/579,830

(22) PCT Filed: Jul. 14, 2022
(86) PCT No.: PCT/US22/37171

§ 371 Date: Jan. 16, 2024

Related U.S. Application Data
(60) Provisional application No. 63/222,852, filed on Jul. 16, 2021.

(51) Int. Cl. C07D 487/04; A61K 31/502; A61P 35/00

(52) U.S. Cl. CPC C07D 487/04; A61K 31/502; A61P 35/00

(57) ABSTRACT

The present invention concerns phthalazinone-based compounds of that inhibit the PARP-1 protein, the compounds having Formula (I):

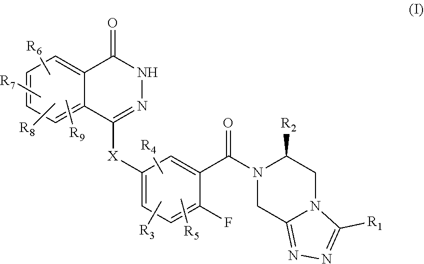

### CROSS-REFERENCE TO RELATED APPLICATIONS

This is the 371 National Phase of International Application No. PCT/US22/37171, filed Jul. 14, 2022, which claims priority to and the benefit of the earlier filing of U.S. Provisional Application No. 63/222,852, filed Jul. 16, 2021; each of which is incorporated by reference herein in its entirety.

### STATEMENT REGARDING FEDERALLY SPONSORED RESEARCH OR DEVELOPMENT

This invention was made with government support under R01 NS088629 awarded by The National Institutes of Health. The government has certain rights in the invention.

### INCORPORATION BY REFERENCE OF SEQUENCE LISTING

The amino acid sequences described herein are shown using standard letter abbreviations, as defined in 37 C.F.R. § 1.822. A computer readable text file, entitled “O046-6000US_SeqList.xml” created on or about Jan. 16, 2024, with a file size of 6,328 bytes, contains the Sequence Listing for this application and is hereby incorporated by reference in its entirety.

### FIELD OF THE INVENTION

The present invention concerns novel phthalazinone-based compounds that inhibit the PARP-1 protein and are useful in methods of treatment including cancers associated with PARP-1 activity.

### BACKGROUND OF THE INVENTION

Poly(ADP-ribose) polymerase 1 (PARP-1) is a multidomain enzyme that uses nicotinamide adenine dinucleotide (NAD⁺) as a substrate. NAD⁺ binding and activation of PARP-1 is mediated by allosteric coupling between the DNA binding domain and the NAD⁺-binding pocket. This allosteric regulation is bidirectional such that certain NAD⁺-competitive inhibitors can increase the affinity of PARP-1 for DNA, a phenomenon referred to as type I inhibition. The structural features that give rise to type I inhibition are incompletely understood. Here, we examined the in vitro and cellular activity of two structurally similar phthalazinone-based, NAD⁺-competitive inhibitors, olaparib and AZ0108, of PARP-1. Unlike olaparib, we found that AZ0108, (S)-4-(difluoro(3-(6-methyl-3-(trifluoromethyl)-5,6,7,8-tetrahydro-[1,2,4]triazolo[4,3-a]pyrazine-7-carbonyl)phenyl)methyl)phthalazin-1(2H)-one, is a type I inhibitor. AZ0108 induced pan-nuclear H2AX phosphorylation and S phase arrest in unperturbed cells. Modeling and mutagenesis studies showed that AZ0108 disrupts interdomain interactions between the autoinhibitory helical domain and the adenine sub-pocket in the catalytic domain. Synthesis of a series of phthalazinone triazolo[4,3-a]pyrazines (Pips) revealed that type I inhibition is influenced by substituent that occupy the adenine sub-pocket. An isosteric analogue of AZ0108, Pip6, showed similar type I and catalytic inhibition of PARP-1 in vitro, yet was ˜80-fold more cytotoxic than AZ0108 in Ewing sarcoma cells. Washout and in-cell competition labeling experiments revealed that Pip6 has a much longer target residence time on PARP-1 compared with AZ0108. Thus the cytoxicity of phthalazinone-based inhibitors of PARP-1 is driven not only by type I inhibition, but also target residence time. These results have important implications for the design of next generation PARP-1 inhibitors with improved anticancer efficacy, for which there remains a significant need.

### SUMMARY OF THE INVENTION

Four FDA-approved inhibitors of PARP-1 are effective therapeutics in cancer cells that have defects in DNA repair (e.g., BRCA1/2-deficient); however, they have yet to find broad use as single agents in cancers (e.g., Ewing sarcoma) that do not have defects in DNA repair pathways. Here we describe inhibitors of PARP-1 that are mechanistically distinct from clinical PARP-1 inhibitors. These inhibitors increase the affinity of PARP-1 for DNA by reverse allostery (referred to as type I inhibition). The most potent analog Pip6, which has a long residence time on PARP-1 in cells, exhibits sub-nanomolar cytotoxicity in Ewing sarcoma cells. Thus, type I inhibitors with long PARP-1 cellular residence time could potentially be used as a monotherapy in Ewing sarcoma.

Provided is a compound of Formula (I):

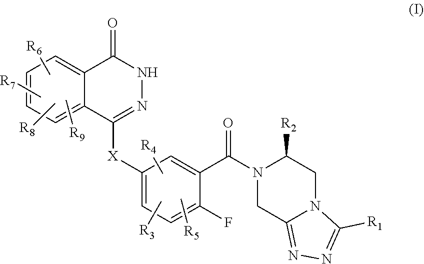

wherein:
X is selected from the group of —CH₂—, —CH(F)—, —C(F)₂—, —O—, —S—, —N(H)—, and —N(C₁-C₄ alkyl)-;
R₁ is selected from the group of C₁-C₄ alkyl, —O—C₁-C₄ alkyl, —S—C₁-C₄ alkyl, C₁-C₄ haloalkyl, —O—C₁-C₄ haloalkyl, —S—C₁-C₄ haloalkyl, C₃-C₆ cycloalkyl, —CH₂—C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl;
with each R₁ C₁-C₄ alkyl, —O—C₁-C₄ alkyl, —S—C₁-C₄ alkyl, C₃-C₆ cycloalkyl, —CH₂—C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl group being optionally substituted by 0, 1, 2, 3, 4, 5, or 6 substituents selected from OH, NH₂, NH(C₁-C₄ alkyl), N(C₁-C₄ alkyl)₂, and halogen; and
R₂ is C₁-C₄ alkyl, optionally substituted by halogen; or, when R₁ is —CH₂—CH₂—CH₃, or —CH₂—CH₂—CH₂—CH₃, R₂ may also be H;
R₃, R₄, R₅, R₆, R₇, and R₈ are each independently selected from the group of H, halogen, C₁-C₄ alkyl, —O—C₁-C₄ alkyl, phenyl, —O-phenyl, benzyl, —O-benzyl, —SO₂—C₁-C₄ alkyl, —SO₂—C₁-phenyl, —SO₂-benzyl, —NH₂, —NH(C₁-C₄ alkyl), —N(C₁-C₄ alkyl)₂, —NH-phenyl, and —NH-benzyl;

wherein the alkyl chains of the R₃, R₄, R₅, R₆, R₇, and R₈ C₁-C₄ alkyl, —O—C₁-C₄ alkyl, —SO₂—C₁-C₄ alkyl, NH(C₁-C₄ alkyl), and —N(C₁-C₄ alkyl)₂ groups may be optionally substituted by 0, 1, 2, 3, 4, or 5 substituents selected from Cl, F, CH₂F, CHF₂, CF₃, CF₂CF₃, OH, CN, NH₂, NH(C₁-C₄ alkyl), N(C₁-C₄ alkyl)₂, and NO₂;
and the rings of the R₃, R₄, R₅, R₆, R₇, and R₈ phenyl, —O-phenyl, benzyl, —O-benzyl, —SO₂—C₁-phenyl, —SO₂-benzyl, —NH-phenyl, and —NH-benzyl groups may be optionally substituted by 0, 1, 2, 3, 4, or 5 substituents selected from C₁-C₄ alkyl, Cl, F, CH₂F, CHF₂, CF₃, CF₂CF₃, OH, CN, NH₂, NH(C₁-C₄ alkyl), N(C₁-C₄ alkyl)₂, and NO₂;
R₉ is selected from the group of H, F, CH₂F, CHF₂, and CF₃;
with the proviso that no more than one of the group of R₃, R₄, and R₅ is phenyl, —O-phenyl, benzyl, —O-benzyl, —SO₂—C₁-phenyl, —SO₂-benzyl, —NH-phenyl, or —NH-benzyl;
with the proviso that no more than one of the group of R₇, R₈, and R₉ is phenyl, —O-phenyl, benzyl, —O-benzyl, —SO₂—C₁-phenyl, —SO₂-benzyl, —NH-phenyl, or —NH-benzyl; and
with the proviso that, when R₂ is CH₃ and each of R₃, R₄, R₅, R₆, R₇, R₈, and R₉ is H, then R₁ is not CF₂CF₃;
or a pharmaceutically acceptable salt, co-crystal, ester, solvate, hydrate, isomer (including optical isomers, racemates, or other mixtures thereof), tautomer, isotope, or polymorph thereof.

### BRIEF DESCRIPTION OF THE MANY VIEWS OF THE DRAWINGS

FIG. 1A presents a theoretical representation of PARP-1-DNA binding allostery.

FIG. 1B presents chemical structure of PARP-1 inhibitors, Olaparib and AZ0108.

FIG. 1C presents a line graph of PARP-1-DNA release over time.

FIG. 1D presents a bar graph of the K_{d} of PARP-1 for a DNA dumbbell probe containing a central nick (5 nM) measured in a fluorescence polarization assay with and without inhibitor (5 μM).

FIG. 2A presents images of HEK293T cells treated with indicated concentration of AZ0108.

FIG. 2B presents a bar graph quantification of mean intensity of γH2AX signal from the panel in FIG. 2A.

FIG. 2C presents a bar graph for measured Mean intensity of γH2AX signal within DAPI masks for HEK293 control or HEK293 PARP-1 KO cells treated with or without 3 μM AZ0108.

FIG. 2D presents a bar graph representing co-treatment with olaparib reversing the effects of AZ0108 on pan-nuclear γH2AX.

FIG. 2E represents inhibition of ATM by KU-55933 partially reduces AZ0108-induced pan-nuclear γH2AX.

FIG. 3A presents a line graph of AZ0108 dose dependent cell growth inhibition in a MTT assay.

FIG. 3B presents a line graph demonstrating that PARP-1 is required for the effects of AZ0108 on cell growth.

FIG. 3C presents a line graph showing olaparib effecting the EC₅₀ value for AZ0108 on cell growth.

FIG. 3D presents a bar graph of the effect of olaparib and AZ0108 on caspase activity.

FIG. 3E presents a bar graph representing the EdU (5-ethynyl-2′-deoxyuridine) incorporation resulting from exposure of HEK 293 cells to agents.

FIG. 4A presents a line graph of D770A PARP-1's affinity for WT PARP-1 DNA.

FIG. 4B presents a bar graph of AZ0108 effects on DNA binding in WT PARP-1 and D770A and R878A mutants.

FIG. 5A presents the structures of synthesized phthalazinone triazolo[4,3-a]pyrazines (Pips).

FIG. 5B presents a line graph representing the effects of Pip1-6 on the binding of PARP-1 to nicked DNA compared to Olaparib and DMSO control.

FIG. 5C presents a bar graph of the effect of Pips on pan-nuclear γH2AX induction.

FIG. 5D presents a line graph representing effects of Pips on HEK 293 cell growth.

FIG. 5E presents an image of western blot showing Pip6 is more potent than AZ0108 in inhibiting PARP-1 PARylation in HEK 293 cells.

FIG. 5F presents a line graph quantifying the western blot images.

### DETAILED DESCRIPTION OF THE INVENTION

For each embodiment described herein there is a further embodiment for the compound wherein all of the variables (X, R₁, R₂, R₃, R₄, R₅, R₆, R₇, R₈, and R₉) are as defined for the embodiment in question, with the further proviso that, when R₂ is CH₃ and each of R₃, R₄, R₅. R₆, R₇, R₈, and R₉ is H, then R₁ is not CH₂F, CHF₂, CF₃, or CF₂CF₃;

Another embodiment provides a compound of Formula (I), or a pharmaceutically acceptable salt, co-crystal, ester, solvate, hydrate, isomer (including optical isomers, racemates, or other mixtures thereof), tautomer, isotope, or polymorph thereof, wherein X is —CH₂— and all other variables (including R₁, R₂, R₃, R₄, R₅, R₆, R₇, R₈, and R₉) are as defined for Formula (I), above.

Another embodiment provides a compound of Formula (I), or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₄ alkyl, —O—C₁-C₄ alkyl, —S—C₁-C₄ alkyl, C₁-C₄ haloalkyl, —O—C₁-C₄ haloalkyl, and —S—C₁-C₄ haloalkyl; and all other variables (including R₂, R₃, R₄, R₅, R₆, R₇, R₈, and R₉) are as defined for Formula (I), above.

Another embodiment provides a compound of Formula (I), or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₃ alkyl, —O—C₁-C₃ alkyl, —S—C₁-C₃ alkyl, C₁-C₃ haloalkyl, —O—C₁-C₃ haloalkyl, and —S—C₁-C₃ haloalkyl; and all other variables (including R₂, R₃, R₄, R₅, R₆, R₇, R₈, and R₉) are as defined for Formula (I), above.

A further embodiment provides a compound of Formula (I), or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; and all other variables (including R₂, R₃, R₄, R₅, R₆, R₇, R₈, and R₉) are as defined for Formula (I), above.

Still another embodiment provides a compound of Formula (I), or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₅ is H; R₆ is H; and all other variables (including R₂, R₃, R₄, R₇, R₈, and R₉) are as defined for Formula (I), above.

An additional embodiment provides a compound of Formula (I), or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₄ is H; R₅ is H; R₆ is H; R₇ is H; and all other variables (including R₂, R₃, R₈, and R₉) are as defined for Formula (I), above.

An additional embodiment provides a compound of Formula (I), or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₃ is H; R₄ is H; R₅ is H; R₆ is H; R₇ is H; and all other variables (including R₂, R₈, and R₉) are as defined for Formula (I), above.

An additional embodiment provides a compound of Formula (II):

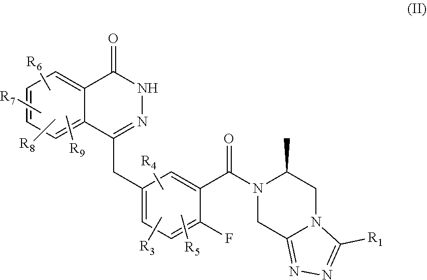

wherein:
R₁ is selected from the group of C₁-C₄ alkyl, —O—C₁-C₄ alkyl, —S—C₁-C₄ alkyl, C₁-C₄ haloalkyl, —O—C₁-C₄ haloalkyl, —S—C₁-C₄ haloalkyl, C₃-C₆ cycloalkyl, —CH₂—C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl;

with each R₁ C₁-C₄ alkyl, —O—C₁-C₄ alkyl, —S—C₁-C₄ alkyl, C₃-C₆ cycloalkyl, —CH₂—C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl group being optionally substituted by 0, 1, 2, 3, 4, 5, or 6 substituents selected from OH, NH₂, NH(C₁-C₄ alkyl), N(C₁-C₄ alkyl)₂, and halogen; and
R₃, R₄, R₅, R₆, R₇, and R₅ are each independently selected from the group of H, halogen, C₁-C₄ alkyl, —O—C₁-C₄ alkyl, phenyl, —O-phenyl, benzyl, —O-benzyl, —SO₂—C₁-C₄ alkyl, —SO₂—C₁-phenyl, —SO₂-benzyl, —NH₂, —NH(C₁-C₄ alkyl), —N(C₁-C₄ alkyl)₂, —NH-phenyl, and —NH-benzyl;
wherein the alkyl chains of the R₃, R₄, R₅, R₆, R₇, and R₈ C₁-C₄ alkyl, —O—C₁-C₄ alkyl, —SO₂-C₁-C₄ alkyl, NH(C₁-C₄ alkyl), and —N(C₁-C₄ alkyl)₂ groups may be optionally substituted by 0, 1, 2, 3, 4, or 5 substituents selected from Cl, F, CH₂F, CHF₂, CF₃, CF₂CF₃, OH, CN, NH₂, NH(C₁-C₄ alkyl), N(C₁-C₄ alkyl)₂, and NO₂;
and the rings of the R₃, R₄, R₅, R₆, R₇, and R₈ phenyl, —O-phenyl, benzyl, —O-benzyl, —SO₂—C₁-phenyl, —SO₂-benzyl, —NH-phenyl, and —NH-benzyl groups may be optionally substituted by 0, 1, 2, 3, 4, or 5 substituents selected from C₁-C₄ alkyl, Cl, F, CH₂F, CHF₂, CF₃, CF₂CF₃, OH, CN, NH₂, NH(C₁-C₄ alkyl), N(C₁-C₄ alkyl)₂, and NO₂;
R₉ is selected from the group of H, F, CH₂F, CHF₂, and CF₃;
with the proviso that no more than one of the group of R₃, R₄, and R₅ is phenyl, —O-phenyl, benzyl, —O-benzyl, —SO₂—C₁-phenyl, —SO₂-benzyl, —NH-phenyl, or —NH-benzyl;
with the proviso that no more than one of the group of R₇, R₈, and R₉ is phenyl, —O-phenyl, benzyl, —O-benzyl, —SO₂—C₁-phenyl, —SO₂-benzyl, —NH-phenyl, or —NH-benzyl; and
with the proviso that, when R₂ is CH₃ and each of R₃, R₄, R₅. R₆, R₇, R₈, and R₉ is H, then R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

Another embodiment provides a compound of Formula (II), or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₄ alkyl, —O—C₁-C₄ alkyl, —S—C₁-C₄ alkyl, C₁-C₄ haloalkyl, —O—C₁-C₄ haloalkyl, and —S—C₁-C₄ haloalkyl; and all other variables (including R₃, R₄, R₅, R₆, R₇, R₈, and R₉) are as defined for Formula (II), above.

Another embodiment provides a compound of Formula (II), or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₃ alkyl, —O—C₁-C₃ alkyl, —S—C₁-C₃ alkyl, C₁-C₃ haloalkyl, —O—C₁-C₃ haloalkyl, and —S—C₁-C₃ haloalkyl; and all other variables (including R₃, R₄, R₅, R₆, R₇, R₈, and R₉) are as defined for Formula (II), above.

A further embodiment provides a compound of Formula (II), or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; and all other variables (including R₃, R₄, R₅, R₆, R₇, R₈, and R₉) are as defined for Formula (II), above.

Still another embodiment provides a compound of Formula (II), or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₅ is H; R₆ is H; and all other variables (including R₃, R₄, R₇, R₈, and R₉) are as defined for Formula (II), above.

An additional embodiment provides a compound of Formula (II), or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₄ is H; R₅ is H; R₆ is H; R₇ is H; and all other variables (including R₃, R₈, and R₉) are as defined for Formula (II), above.

An additional embodiment provides a compound of Formula (II), or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₃ is H; R₄ is H; R₅ is H; R₆ is H; R₇ is H; and all other variables (including R₈ and R₉) are as defined for Formula (II), above.

A further embodiment provides a compound of Formula (III):

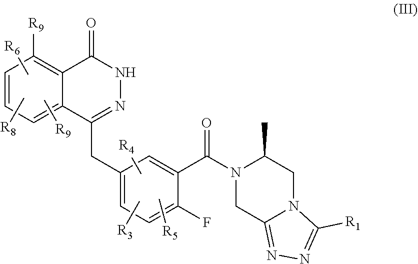

wherein:
R₁ is selected from the group of C₁-C₄ alkyl, —O—C₁-C₄ alkyl, —S—C₁-C₄ alkyl, C₁-C₄ haloalkyl, —O—C₁-C₄ haloalkyl, —S—C₁-C₄ haloalkyl, C₃-C₆ cycloalkyl, —CH₂-C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl;
with each R₁ C₁-C₄ alkyl, —O—C₁-C₄ alkyl, —S—C₁-C₄ alkyl, C₃-C₆ cycloalkyl, —CH₂-C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl group being optionally substituted by 0, 1, 2, 3, 4, 5, or 6 substituents selected from OH, NH₂, NH(C₁-C₄ alkyl), N(C₁-C₄ alkyl)₂, and halogen; and
R₃, R₄, R₅, R₆, R₇, and R₅ are each independently selected from the group of H, halogen, C₁-C₄ alkyl, —O—C₁-C₄ alkyl, phenyl, —O-phenyl, benzyl, —O-benzyl, —SO₂—C₁-C₄ alkyl, —SO₂—C₁-phenyl, —SO₂-benzyl, —NH₂, —NH(C₁-C₄ alkyl), —N(C₁-C₄ alkyl)₂, —NH-phenyl, and —NH-benzyl;
wherein the alkyl chains of the R₃, R₄, R₅, R₆, R₇, and R₈ C₁-C₄ alkyl, —O—C₁-C₄ alkyl, —SO₂-C₁-C₄ alkyl, NH(C₁-C₄ alkyl), and —N(C₁-C₄ alkyl)₂ groups may be optionally substituted by 0, 1, 2, 3, 4, or 5 substituents selected from Cl, F, CH₂F, CHF₂, CF₃, CF₂CF₃, OH, CN, NH₂, NH(C₁-C₄ alkyl), N(C₁-C₄ alkyl)₂, and NO₂;
and the rings of the R₃, R₄, R₅, R₆, R₇, and R₈ phenyl, —O-phenyl, benzyl, —O-benzyl, —SO₂-C₁-phenyl, —SO₂-benzyl, —NH-phenyl, and —NH-benzyl groups may be optionally substituted by 0, 1, 2, 3, 4, or 5 substituents selected from C₁-C₄ alkyl, Cl, F, CH₂F, CHF₂, CF₃, CF₂CF₃, OH, CN, NH₂, NH(C₁-C₄ alkyl), N(C₁-C₄ alkyl)₂, and NO₂;
R₉ is selected from the group of H, F, CH₂F, CHF₂, and CF₃;
with the proviso that no more than one of the group of R₃, R₄, and R₅ is phenyl, —O-phenyl, benzyl, —O-benzyl, —SO₂—C₁-phenyl, —SO₂-benzyl, —NH-phenyl, or —NH-benzyl;
with the proviso that no more than one of the group of R₇, R₈, and R₉ is phenyl, —O-phenyl, benzyl, —O-benzyl, —SO₂—C₁-phenyl, —SO₂-benzyl, —NH-phenyl, or —NH-benzyl; and
with the proviso that, when R₂ is CH₃ and each of R₃, R₄, R₅. R₆, R₇, R₈, and R₉ is H, then R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

Another embodiment provides a compound of Formula (III), or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₄ alkyl, —O—C₁-C₄ alkyl, —S—C₁-C₄ alkyl, C₁-C₄ haloalkyl, —O—C₁-C₄ haloalkyl, and —S—C₁-C₄ haloalkyl; and all other variables (including R₃, R₄, R₅, R₆, R₇, R₈, and R₉) are as defined for Formula (III), above.

Another embodiment provides a compound of Formula (III), or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₃ alkyl, —O—C₁-C₃ alkyl, —S—C₁-C₃ alkyl, C₁-C₃ haloalkyl, —O—C₁-C₃ haloalkyl, and —S—C₁-C₃ haloalkyl; and all other variables (including R₃, R₄, R₅, R₆, R₇, R₈, and R₉) are as defined for Formula (III), above.

A further embodiment provides a compound of Formula (III), or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; and all other variables (including R₃, R₄, R₅, R₆, R₇, R₈, and R₉) are as defined for Formula (III), above.

Still another embodiment provides a compound of Formula (III), or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₅ is H; R₆ is H; and all other variables (including R₃, R₄, R₇, R₈, and R₉) are as defined for Formula (III), above.

An additional embodiment provides a compound of Formula (III), or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₄ is H; R₅ is H; R₆ is H; R₇ is H; and all other variables (including R₃, R₈, and R₉) are as defined for Formula (III), above.

An additional embodiment provides a compound of Formula (III), or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₃ is H; R₄ is H; R₅ is H; R₆ is H; R₇ is H; and all other variables (including R₈ and R₉) are as defined for Formula (III), above.

Yet another embodiment provides a compound of Formula (IV):

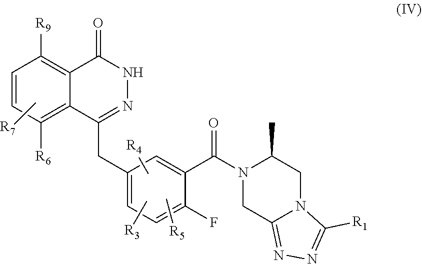

wherein:
R₁ is selected from the group of C₁-C₄ alkyl, —O—C₁-C₄ alkyl, —S—C₁-C₄ alkyl, C₁-C₄ haloalkyl, —O—C₁-C₄ haloalkyl, —S—C₁-C₄ haloalkyl, C₃-C₆ cycloalkyl, —CH₂-C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl;
with each R₁ C₁-C₄ alkyl, —O—C₁-C₄ alkyl, —S—C₁-C₄ alkyl, C₃-C₆ cycloalkyl, —CH₂-C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl group being optionally substituted by 0, 1, 2, 3, 4, 5, or 6 substituents selected from OH, NH₂, NH(C₁-C₄ alkyl), N(C₁-C₄ alkyl)₂, and halogen; and
R₃, R₄, R₅, R₆, R₇, and R₅ are each independently selected from the group of H, halogen, C₁-C₄ alkyl, —O—C₁-C₄ alkyl, phenyl, —O-phenyl, benzyl, —O-benzyl, —SO₂—C₁-C₄ alkyl, —SO₂—C₁-phenyl, —SO₂-benzyl, —NH₂, —NH(C₁-C₄ alkyl), —N(C₁-C₄ alkyl)₂, —NH-phenyl, and —NH-benzyl;
wherein the alkyl chains of the R₃, R₄, R₅, R₆, R₇, and R₈ C₁-C₄ alkyl, —O—C₁-C₄ alkyl, —SO₂-C₁-C₄ alkyl, NH(C₁-C₄ alkyl), and —N(C₁-C₄ alkyl)₂ groups may be optionally substituted by 0, 1, 2, 3, 4, or 5 substituents selected from Cl, F, CH₂F, CHF₂, CF₃, CF₂CF₃, OH, CN, NH₂, NH(C₁-C₄ alkyl), N(C₁-C₄ alkyl)₂, and NO₂;
and the rings of the R₃, R₄, R₅, R₆, R₇, and R₈ phenyl, —O-phenyl, benzyl, —O-benzyl, —SO₂-C₁-phenyl, —SO₂-benzyl, —NH-phenyl, and —NH-benzyl groups may be optionally substituted by 0, 1, 2, 3, 4, or 5 substituents selected from C₁-C₄ alkyl, Cl, F, CH₂F, CHF₂, CF₃, CF₂CF₃, OH, CN, NH₂, NH(C₁-C₄ alkyl), N(C₁-C₄ alkyl)₂, and NO₂;
R₉ is selected from the group of H, F, CH₂F, CHF₂, and CF₃;
with the proviso that no more than one of the group of R₃, R₄, and R₅ is phenyl, —O-phenyl, benzyl, —O-benzyl, —SO₂—C₁-phenyl, —SO₂-benzyl, —NH-phenyl, or —NH-benzyl;
with the proviso that no more than one of the group of R₇, R₈, and R₉ is phenyl, —O-phenyl, benzyl, —O-benzyl, —SO₂—C₁-phenyl, —SO₂-benzyl, —NH-phenyl, or —NH-benzyl; and
with the proviso that, when R₂ is CH₃ and each of R₃, R₄, R₅. R₆, R₇, R₈, and R₉ is H, then R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

Another embodiment provides a compound of Formula (IV), or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₄ alkyl, —O—C₁-C₄ alkyl, —S—C₁-C₄ alkyl, C₁-C₄ haloalkyl, —O—C₁-C₄ haloalkyl, and —S—C₁-C₄ haloalkyl; and all other variables (including R₃, R₄, R₅, R₆, R₇, and R₉) are as defined for Formula (IV), above.

Another embodiment provides a compound of Formula (IV), or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₃ alkyl, —O—C₁-C₃ alkyl, —S—C₁-C₃ alkyl, C₁-C₃ haloalkyl, —O—C₁-C₃ haloalkyl, and —S—C₁-C₃ haloalkyl; and all other variables (including R₃, R₄, R₅, R₆, R₇, and R₉) are as defined for Formula (IV), above.

A further embodiment provides a compound of Formula (IV), or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; and all other variables (including R₃, R₄, R₅, R₆, R₇, and R₉) are as defined for Formula (IV), above.

Still another embodiment provides a compound of Formula (IV), or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₅ is H; R₆ is H; and all other variables (including R₃, R₄, R₇, and R₉) are as defined for Formula (IV), above.

An additional embodiment provides a compound of Formula (IV), or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₄ is H; R₅ is H; R₆ is H; R₇ is H; and all other variables (including R₃, and R₉) are as defined for Formula (IV), above.

An additional embodiment provides a compound of Formula (IV), or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₃ is H; R₄ is H; R₅ is H; R₆ is H; R₇ is H; and all other variables (including R₉) are as defined for Formula (IV), above.

A further embodiment provides a compound of Formula (V):

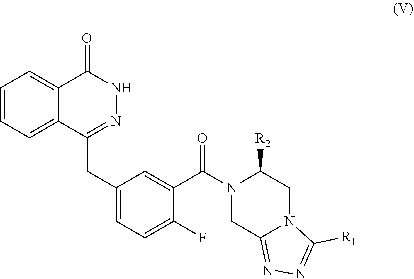

wherein:
R₁ is selected from the group of C₁-C₄ alkyl, —O—C₁-C₄ alkyl, —S—C₁-C₄ alkyl, C₁-C₄ haloalkyl, —O—C₁-C₄ haloalkyl, —S—C₁-C₄ haloalkyl, C₃-C₆ cycloalkyl, —CH₂-C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl;
with each R₁ C₁-C₄ alkyl, —O—C₁-C₄ alkyl, —S—C₁-C₄ alkyl, C₃-C₆ cycloalkyl, —CH₂-C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl group being optionally substituted by 0, 1, 2, 3, 4, 5, or 6 substituents selected from OH, NH₂, NH(C₁-C₄ alkyl), N(C₁-C₄ alkyl)₂, and halogen;
R₂ is C₁-C₄ alkyl; or, when R₁ is —CH₂—CH₂—CH₃, or —CH₂—CH₂—CH₂—CH₃, R₂ may also be H;
with the proviso that, when R₂ is CH₃, R₁ is not CF₂CF₃;
or a pharmaceutically acceptable salt, co-crystal, ester, solvate, hydrate, isomer (including optical isomers, racemates, or other mixtures thereof), tautomer, isotope, or polymorph thereof.

Another embodiment provides a compound of Formula (V), wherein:
R₁ is selected from the group of C₁-C₄ haloalkyl, C₃-C₄ cycloalkyl, —CH₂-C₃-C₄ cycloalkyl, and C₁-C₃ alkyl, wherein the R₁ C₃-C₄ cycloalkyl, —CH₂-C₃-C₄ cycloalkyl, and C₁-C₃ alkyl groups are optionally substituted by substituents selected from OH, NH₂, and halogen; and
R₂ is C₁-C₄ alkyl; or, when R₁ is —CH₂—CH₂—CH₃, R₂ may also be H;
with the proviso that, when R₂ is CH₃, R₁ is not CF₂CF₃;
or a pharmaceutically acceptable salt, co-crystal, ester, solvate, hydrate, isomer (including optical isomers, racemates, or other mixtures thereof), tautomer, isotope, or polymorph thereof.

A different embodiment provides a compound of Formula (V), wherein:
R₁ is selected from the group of C₁-C₃ haloalkyl, C₃-C₄ cycloalkyl, —CH₂—C₃-C₄ cycloalkyl, and C₁-C₃ alkyl, wherein the R₁ C₃-C₄ cycloalkyl, —CH₂—C₃-C₄ cycloalkyl, and C₁-C₃ alkyl groups are optionally substituted by substituents selected from OH, NH₂, and halogen; and
R₂ is C₁-C₄ alkyl; or, when R₁ is —CH₂—CH₂—CH₃, R₂ may also be H;
with the proviso that, when R₂ is CH₃, R₁ is not CF₂CF₃;
or a pharmaceutically acceptable salt, co-crystal, ester, solvate, hydrate, isomer (including optical isomers, racemates, or other mixtures thereof), tautomer, isotope, or polymorph thereof.

Further provided is a compound of Formula (VI):

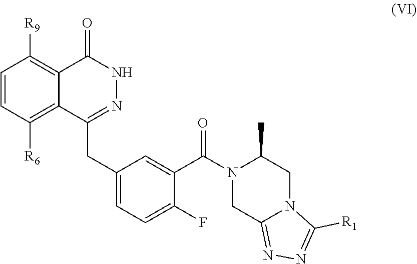

wherein,
R₁ is C₁-C₄ haloalkyl;
R₆ is selected from the group of H, halogen, C₁-C₄ alkyl, —O—C₁-C₄ alkyl, phenyl, —O-phenyl, benzyl, —O-benzyl, —SO₂—C₁-C₄ alkyl, —SO₂—C₁-phenyl, —SO₂-benzyl, —NH₂, —NH(C₁-C₄ alkyl), —N(C₁-C₄ alkyl)₂, —NH-phenyl, and —NH-benzyl;
wherein the alkyl chains of the R₆ C₁-C₄ alkyl, —O—C₁-C₄ alkyl, —SO₂—C₁-C₄ alkyl, NH(C₁-C₄ alkyl), and —N(C₁-C₄ alkyl)₂ groups may be optionally substituted by 0, 1, 2, 3, 4, or 5 substituents selected from Cl, F, CH₂F, CHF₂, CF₃, CF₂CF₃, OH, CN, NH₂, NH(C₁-C₄ alkyl), N(C₁-C₄ alkyl)₂, and NO₂;
and the rings of the R₆ phenyl, —O-phenyl, benzyl, —O-benzyl, —SO₂—C₁-phenyl, —SO₂-benzyl, —NH-phenyl, and —NH-benzyl groups may be optionally substituted by 0, 1, 2, 3, 4, or 5 substituents selected from C₁-C₄ alkyl, Cl, F, CH₂F, CHF₂, CF₃, CF₂CF₃, OH, CN, NH₂, NH(C₁-C₄ alkyl), N(C₁-C₄ alkyl)₂, and NO₂;
R₉ is selected from the group of H, F, CH₂F, CHF₂, and CF₃;
with the proviso that, when R₆ is H and Ry is H, then R₁ is not CF₂CF₃;
or a pharmaceutically acceptable salt, co-crystal, ester, solvate, hydrate, isomer (including optical isomers, racemates, or other mixtures thereof), tautomer, isotope, or polymorph thereof.

Another embodiment provides a compound of Formula (VI), wherein:
R₁ is C₁-C₄ haloalkyl;
R₆ is selected from the group of H, halogen, C₁-C₄ alkyl, —O—C₁-C₄ alkyl, —NH₂, —NH(C₁-C₄ alkyl), and —N(C₁-C₄ alkyl)₂;
wherein the alkyl chains of the R₆ C₁-C₄ alkyl, —O—C₁-C₄ alkyl, NH(C₁-C₄ alkyl), and —N(C₁-C₄ alkyl)₂ groups may be optionally substituted by 0, 1, 2, 3, 4, or 5 substituents selected from Cl, F, CH₂F, CHF₂, CF₃, CF₂CF₃, OH, CN, NH₂, NH(C₁-C₄ alkyl), N(C₁-C₄ alkyl)₂, and NO₂;
R₉ is selected from the group of H, F, CH₂F, CHF₂, and CF₃;
with the proviso that, when R₆ is H and R₉ is H, then R₁ is not CF₂CF₃;
or a pharmaceutically acceptable salt, co-crystal, ester, solvate, hydrate, isomer (including optical isomers, racemates, or other mixtures thereof), tautomer, isotope, or polymorph thereof.

A different embodiment provides a compound of Formula (VI), wherein:
R₁ is C₁-C₃ haloalkyl;
R₆ is selected from the group of H, halogen, C₁-C₄ alkyl, —O—C₁-C₄ alkyl, —NH₂, —NH(C₁-C₄ alkyl), and —N(C₁-C₄ alkyl)₂;
wherein the alkyl chains of the R₆ C₁-C₄ alkyl, —O—C₁-C₄ alkyl, NH(C₁-C₄ alkyl), and —N(C₁-C₄ alkyl)₂ groups may be optionally substituted by 0, 1, 2, 3, 4, or 5 substituents selected from Cl, F, CH₂F, CHF₂, CF₃, CF₂CF₃, OH, CN, NH₂, NH(C₁-C₄ alkyl), N(C₁-C₄ alkyl)₂, and NO₂;
R₉ is selected from the group of H, F, CH₂F, CHF₂, and CF₃;
with the proviso that, when R₆ is H and Ry is H, then R₁ is not CF₂CF₃;
or a pharmaceutically acceptable salt, co-crystal, ester, solvate, hydrate, isomer (including optical isomers, racemates, or other mixtures thereof), tautomer, isotope, or polymorph thereof.

A further embodiment provides a compound of Formula (VII):

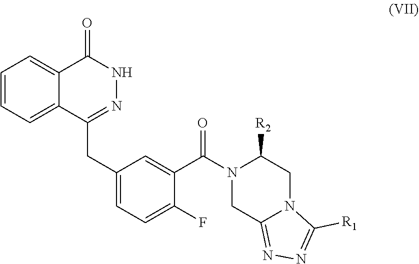

wherein:
R₁ is selected from the group of C₃-C₆ cycloalkyl, —CH₂—C₃-C₆ cycloalkyl, and C₁-C₄ alkyl optionally substituted by substituents selected from OH, NH₂, and halogen; and
R₂ is C₁-C₄ alkyl; or, when R₁ is —CH₂—CH₂—CH₃, or —CH₂—CH₂—CH₂—CH₃, R₂ may also be H;
with the proviso that, when R₂ is CH₃, R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

A further embodiment provides a compound of Formula (VII), as shown above, wherein:
R₁ is selected from the group of C₃-C₄ cycloalkyl, —CH₂—C₃-C₄ cycloalkyl, and C₁-C₃ alkyl optionally substituted by substituents selected from OH, NH₂, and halogen; and
R₂ is C₁-C₄ alkyl; or, when R₁ is —CH₂—CH₂—CH₃, R₂ may also be H;
with the proviso that, when R₂ is CH₃, R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

Another embodiment provides a compound of Formula (VII), as shown above, wherein:
R₁ is C₁-C₃ alkyl optionally substituted by substituents selected from OH, NH₂, and halogen; and
R₂ is C₁-C₄ alkyl; or, when R₁ is —CH₂—CH₂—CH₃, R₂ may also be H;
with the proviso that, when R₂ is CH₃, R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

Another embodiment provides a compound of Formula (VII), as shown above, wherein:
R₁ is C₁-C₃ alkyl optionally substituted by substituents selected from OH, NH₂, and F; and
R₂ is C₁-C₄ alkyl; or, when R₁ is —CH₂—CH₂—CH₃, R₂ may also be H;
with the proviso that, when R₂ is CH₃, R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

Another embodiment provides a compound of Formula (VII), as shown above, wherein:
R₁ is C₁-C₃ alkyl optionally substituted by one or more F substituents; and
R₂ is C₁-C₃ alkyl; or, when R₁ is —CH₂—CH₂—CH₃, R₂ may also be H;
with the proviso that, when R₂ is CH₃, R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

Another embodiment provides a compound of Formula (VII), as shown above, wherein:
R₁ is C₁-C₃ alkyl optionally substituted by one or more F substituents; and
R₂ is CH₃;
with the proviso that, when R₂ is CH₃, R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

Another embodiment provides a compound of Formula (VII), as shown above, wherein: R₁ is selected from the group of —CHF—CH₃, —CF₂—CH₃, —CH₂—CH₂F, —CH₂—CHF₂, —CH₂—CF₃, and —CHF—CF₃; and
R₂ is CH₃;
or a pharmaceutically acceptable salt, co-crystal, ester, solvate, hydrate, isomer (including optical isomers, racemates, or other mixtures thereof), tautomer, isotope, or polymorph thereof.

Another embodiment provides a compound of Formula (VII), as shown above, wherein:
R₁ is selected from the group of —CHF—CH₃ and —CF₂—CH₃; and
R₂ is CH₃;
or a pharmaceutically acceptable salt, co-crystal, ester, solvate, hydrate, isomer (including optical isomers, racemates, or other mixtures thereof), tautomer, isotope, or polymorph thereof.

The compounds described herein may be used in the treatment or amelioration of diseases or conditions associated with PARP-1 expression, particularly in those diseases or conditions associated with excess PARP-1 expression or activity.

Such uses include those in methods of treating various cancers, including, but not limited to, ovarian cancer, deleterious BRCA mutation (germline and/or somatic)-associated epithelial ovarian cancer, fallopian tube cancer, cervical cancer (including recurrent cervical cancer), or primary peritoneal cancer (including those in a complete or partial response to platinum-based chemotherapy, as well as those in subjects who have been treated with two or more chemotherapies), stomach cancer, prostate cancer including metastatic castration-resistant prostate cancer), lymphomas, melanomas, breast cancer (including triple negative breast cancer and HER2+ breast cancer), lung cancer, Ewing sarcoma, osteosarcoma, glioblastoma, lymphoma, skin cancer, kidney cancer, leukemia (including Acute Myeloid Leukemia), testicular cancer, pancreatic cancer (including pancreatic ductal adenocarcinoma, pancreatic acinar cell carcinoma, pancreatic neuroendocrine tumors, pancreatic squamous cell carcinoma, pancreatic adenosquamous carcinoma, and pancreatic colloid carcinoma), and other neoplastic diseases.

The PARP-1 inhibiting compounds herein may be used in methods of treatment of lung cancer, including BRCA mutant lung cancer, small cell lung cancer, non-small cell lung cancer, Ewing sarcoma, lung carcinoma,

The PARP-I inhibiting compounds (PARP-1 inhibitors) described herein may also be used in the treatment (including prophylactic treatment), modulation, or amelioration of other diseases or conditions associated with PARP-1 expression and activity, including inflammatory pathways, diabetic kidney disease, heart failure, cardiomyopathies, circulatory shock, cardiovascular aging, diabetic cardiovascular complications, myocardial hypertrophy, atherosclerosis, vascular remodeling following injury, aortic aneurysms, and angiogenesis.

PARP-1 activation has also been associated with neurodegenerative conditions, including Alzheimer's disease and Parkinson's disease.

In each instance, the method of treatment comprises administering to the subject in need thereof a therapeutically effective amount of a PARP-1 inhibiting compound as described herein, or a pharmaceutically acceptable salt, co-crystal, ester, solvate, hydrate, isomer (including optical isomers, racemates, or other mixtures thereof), tautomer, isotope, or polymorph thereof.

### Definitions

The term “alkyl” refers to a straight or branched hydrocarbon. For example, an alkyl group can have 1 to 4 carbon atoms (i.e, C₁-C₄ alkyl), 1 to 3 carbon atoms (i.e., C₁-C₃ alkyl), or 1 to 2 carbon atoms (i.e., C₁-C₂ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), and 2-methyl-2-propyl (t-Bu, t-butyl, —C(CH₃)₃).

The term “haloalkyl” refers to an alkyl group, as defined above, in which one or more hydrogen atoms of the alkyl group is replaced with a halogen atom. The alkyl portion of a haloalkyl group can have, for instance, 1 to 4 carbon atoms (i.e., C₁-C₄ haloalkyl), 1 to 3 carbon atoms (i.e., C₁-C₃ haloalkyl), or 1 to 2 carbon atoms (i.e., C₁-C₂ haloalkyl). Non-limiting examples of suitable haloalkyl groups, which may also be referred to as halofluoro groups include, but are not limited to, trifluoromethyl (—CF₃), difluoromethyl (—CHF₂), fluoromethyl (—CFH₂), 2-fluoroethyl (—CH₂CH₂F), 2-fluoropropyl (—CH₂CHF₂), 2,2,2-trifluoroetheyl (—CH₂CF₃), 1,1-difluoroethyl (—CF₂CH₃), 2-fluoropropyl (—CH₂CHFCH₃), 1,1-difluoropropyl (—CF₂CH₂CH₃), 2,2-difluoropropyl (—CH₂CF₂CH₃), 3,3-difluoropropyl (—CH₂CH₂CHF₂), 3,3,3-trifluoropropyl (—CH₂CH₂CHF₃), 1,1-difluorobutyl (—CF₂CH₂CH₂CH₃), perfluoroethyl (—CF₂CF₃), perfluoropropyl (—CF₂CF₂CF₃), 1,1,2,2,3,3-hexafluorobutyl (—CF₂—CF₂CF₂CH₃), perfluorobutyl (—CF₂CF₂CF₂CF₃), 1,1,1,3,3,3-hexafluoropropan-2-yl (—CH₂(CF₃)₂) groups, and the like. For each embodiment described herein using the term “haloalkyl,” there is a further embodiment in which all other variables (such as X and R₁-R₉) are as defined for the initial embodiment in question, except the haloalkyl variable (such as C₁-C₄ haloalkyl, —O—C₁-C₄ haloalkyl, or —S—C₁-C₄ haloalkyl is a fluoroalkyl having the same number of carbon atoms. Additional haloalkyl groups wherein the halogen substitution is with bromine, iodine, or chlorine atoms are also understood for use herein.

A “heterocycle,” “heterocyclyl group,” or “heterocyclic group” herein refers to a chemical ring containing carbon atoms and at least one ring heteroatom selected from O, S, and N. Examples of 5-membered and 6-membered heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, 2,2-dimethyl-1,3-dioxolanyl, 4-piperidinyl, pyridinyl, pyrrolidinyl, 2-pyrrolidonyl, pyrrolinyl, tetrahydrofuranyl, triazinyl, 6H-1,2,5-thiadiazinyl, 2H,6H-1,5,2-dithiazinyl, thienyl, thianthrenyl, pyranyl, 2H-pyrrolyl, isothiazolyl, isoxazolyl, pyrazinyl, pyridazinyl, imidazolidinyl, imidazolinyl, pyrazolidinyl, pyrazolinyl, piperazinyl, morpholinyl, and oxazolidinyl.

The terms “therapeutically effective amount” or “pharmaceutically effective amount” as used herein refer to an amount that is sufficient to effect treatment, as defined below, when administered to a subject (e.g., a mammal, such as a human) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, a “therapeutically effective amount” or a “pharmaceutically effective amount” of a compound of Formula I, or a pharmaceutically acceptable salt or co-crystal thereof, is an amount sufficient to modulate PARP-1 expression or activity, and thereby treat a subject (e.g., a human) suffering an indication, or to ameliorate or alleviate the existing symptoms of the indication. For example, a therapeutically or pharmaceutically effective amount may be an amount sufficient to decrease a symptom of a disease or condition responsive to inhibition of PARP-1 activity. Ideally, an effective amount of an agent is an amount sufficient to inhibit or treat the disease without causing substantial toxicity in the subject. The effective amount of an agent will be dependent on the subject being treated, the severity of the affliction, and the manner of administration of the pharmaceutical composition. Methods of determining an effective amount of the disclosed compound sufficient to achieve a desired effect in a subject will be understood by those of skill in the art in light of this disclosure. In some embodiments, a therapeutically effective amount may be administered to an adult human subject at a dose of from about 1 mg to about 1,000 mg per day in one dose or divided into two doses. In some embodiments, the compound in question may be administered to an adult subject at a dose of from about 50 mg to about 500 mg once or twice per day. In other separate embodiments, the compound in question may be administered to an adult subject at a dose, respectively, of from about 1 mg to about 100 mg, of from about 50 mg to about 400 mg, about 50 mg to about 300 mg, about 50 mg to about 250 mg, and about 50 mg to about 200 mg, each dose given once or twice per day. In some embodiments, the individual dose is selected from the group of 1 mg, 5 mg, 10 mg, 20 mg, 30 mg, 40 mg, 50 mg, 60 mg, 75 mg, 100 mg, 150 mg, 200 mg, 250 mg, 300 mg, 350 mg, 400 mg, and 500 mg per dose.

“Subject” refers to an animal, such as a mammal, that has been or will be the object of treatment, observation, or experiment. The methods described herein may be useful in both human therapy and veterinary applications. In some embodiments, the subject is a mammal; in some embodiments the subject is human; and in some embodiments the subject is chosen from cats and dogs. “Subject in need thereof” or “human in need thereof” refers to a subject, such as a human, who may have or is suspected to have diseases or conditions that would benefit from certain treatment; for example, treatment with a compound of Formula I, or a pharmaceutically acceptable salt or co-crystal thereof, as described herein. This includes a subject who may be determined to be at risk of or susceptible to such diseases or conditions, such that treatment would prevent the disease or condition from developing.

“Treatment” or “treating” is an approach for obtaining beneficial or desired results including clinical results. Beneficial or desired clinical results may include one or more of the following: (i) inhibiting the disease or condition (e.g., decreasing one or more symptoms resulting from the disease or condition, and/or diminishing the extent of the disease or condition); (ii) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (iii) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

The terms “inhibiting” or “inhibition” indicates a decrease, such as a significant decrease, in the baseline activity of a biological activity or process. “Inhibition of PARP-1 activity” refers to a decrease in PARP-1 activity as a direct or indirect response to the presence of a compound of Formula I, or a pharmaceutically acceptable salt or co-crystal thereof, relative to the activity of PARP-1 in the absence of such compound or a pharmaceutically acceptable salt or co-crystal thereof. The decrease in activity may be due to the direct interaction of the compound with PARP-1, or due to the interaction of the compound(s) described herein with one or more other factors that in turn affect PARP-1 activity. For example, the presence of the compound(s) may decrease PARP-1 activity by directly binding to the PARP-1, by causing (directly or indirectly) another factor to decrease PARP-1 activity, or by (directly or indirectly) decreasing the amount of PARP-1 present in the cell or organism. In some embodiments, the inhibition of PARP-1 activity may be compared in the same subject prior to treatment, or other subjects not receiving the treatment. The term “inhibitor” is understood to refer to a compound or agent that, upon administration to a human in need thereof at a pharmaceutically or therapeutically effective dose, provides the inhibition activity desired.

“Delaying” the development of a disease or condition means to defer, hinder, slow, retard, stabilize, and/or postpone development of the disease or condition. This delay can be of varying lengths of time, depending on the history of the disease or condition, and/or subject being treated. A method that “delays” development of a disease or condition is a method that reduces probability of disease or condition development in a given time frame and/or reduces the extent of the disease or condition in a given time frame, when compared to not using the method. Such comparisons are typically based on clinical studies, using a statistically significant number of subjects. Disease or condition development can be detectable using standard methods, such as routine physical exams, mammography, imaging, or biopsy. Development may also refer to disease or condition progression that may be initially undetectable and includes occurrence, recurrence, and onset.

By “significant” is meant any detectable change that is statistically significant in a standard parametric test of statistical significance such as Student's T-test, where p<0.05.

The term “level of expression” herein refers to the rate of processing information from a gene in the synthesis of a gene product, particularly of a functional gene product. In different embodiments gene expression may be indicated by transcriptional expression of mRNA levels or protein levels.

In some embodiments the various methods of treatment, the PARP-1 inhibitors described herein may be used in combination with standard of care treatment(s) for the disease or condition in question. In other embodiments, the PARP-1 inhibitors described herein may be used in combination with other PARP-1 inhibitors, including those selected from the group of rucaparib (RUBACRA®), olaparib, veliparib, iniparib, INO01001, MK4827, CEP-9722, and BMN-673.

The term “pharmaceutically acceptable salt” or “therapeutically acceptable salt” refer to a salt form of a compound of Formula (I) which is, within the scope of sound medical evaluation, suitable for use in contact with the tissues and organs of humans and/or animals such that any resulting toxicity, irritation, allergic response, and the like and are commensurate with a reasonable benefit/risk ratio. “Pharmaceutically acceptable salts” include, for example, salts with inorganic acids and salts with an organic acid. Examples of salts may include hydrochloride, phosphate, diphosphate, hydrobromide, sulfate, sulfinate, nitrate, malate, maleate, fumarate, tartrate, succinate, citrate, acetate, lactate, methanesulfonate (mesylate), benzenesuflonate (besylate), p-toluenesulfonate (tosylate), 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

The term “crystal forms” and related terms herein refer to the various crystalline modifications of a given substance, including, but not limited to, polymorphs, solvates, hydrates, co-crystals, and other molecular complexes, as well as salts, solvates of salts, hydrates of salts, other molecular complexes of salts, and polymorphs thereof. Crystal forms of a substance can be obtained by a number of methods, as known in the art. Such methods include, but are not limited to, melt recrystallization, melt cooling, solvent recrystallization, recrystallization in confined spaces such as, e.g., in nanopores or capillaries, recrystallization on surfaces or templates, such as, e.g., on polymers, recrystallization in the presence of additives, such as, e.g., co-crystal counter-molecules, desolvation, dehydration, rapid evaporation, rapid cooling, slow cooling, vapor diffusion, sublimation, grinding and solvent-drop grinding.

The term “co-crystal” or “co-crystal salt” as used herein means a crystalline material composed of two or more unique solids at room temperature, each of which has distinctive physical characteristics such as structure, melting point, and heats of fusion, hygroscopicity, solubility, and stability. A co-crystal or a co-crystal salt can be produced according to a per se known co-crystallization method. The terms co-crystal (or cocrystal) or co-crystal salt also refer to a multicomponent system in which there exists a host API (active pharmaceutical ingredient) molecule or molecules, such as a compound of Formula I, and a guest (or co-former) molecule or molecules. In particular embodiments the pharmaceutically acceptable co-crystal of the compound of Formula I or of the compound of Formula II with a co-former molecule is in a crystalline form selected from a malonic acid co-crystal, a succinic acid co-crystal, a decanoic acid co-crystal, a salicylic acid co-crystal, a vanillic acid co-crystal, a maltol co-crystal, or a glycolic acid co-crystal. Co-crystals may have improved properties as compared to the parent form (i.e., the free molecule, zwitter ion, etc.) or a salt of the parent compound. Improved properties can include increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

The term “pharmaceutical composition” refers to a composition containing a pharmaceutically effective amount of one or more of the isotopic compounds described herein, or a pharmaceutically acceptable salt thereof, formulated with a pharmaceutically acceptable carrier, which can also include other additives, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein. Conventional procedures and ingredients for the selection and preparation of suitable formulations are described, for example, in Remington: The Science and Practice of Pharmacy, 21^{st} Ed., Gennaro, Ed., Lippencott Williams & Wilkins (2005) and in The United States Pharmacopeia: The National Formulary (USP 36 NF31), published in 2013. Suitable parenteral administration routes include various means of intravascular administrations, including intravenous bolus injections, intravenous infusions, intra-arterial bolus injections, vasculature catheterizations and intra-arterial infusions; peri- and intra-tissue injection, including peri-tumoral and intra-tumoral injections, intra-retinal injections, or subretinal injections; and subcutaneous injections or depositions including subcutaneous infusion by means such as osmotic pumps.

As used herein, “pharmaceutically acceptable excipient” is a pharmaceutically acceptable vehicle that includes, without limitation, any and all carriers, solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “carrier” refers to an excipient or vehicle that includes without limitation diluents, disintegrants, precipitation inhibitors, surfactants, glidants, binders, lubricants, and the like with which the compound is administered. Carriers are generally described herein and also in “Remington's Pharmaceutical Sciences” by E. W. Martin. Examples of carriers include, but are not limited to, aluminum monostearate, aluminum stearate, carboxymethylcellulose, carboxymethylcellulose sodium, crospovidone, glyceryl isostearate, glyceryl monostearate, hydroxyethyl cellulose, hydroxyethyl cellulose, hydroxymethyl cellulose, hydroxyoctacosanyl hydroxystearate, hydroxypropyl cellulose, hydroxypropyl cellulose, hydroxypropyl methylcellulose, lactose, lactose monohydrate, magnesium stearate, mannitol, microcrystalline cellulose, poloxamer 124, poloxamer 181, poloxamer 182, poloxamer 188, poloxamer 237, poloxamer 407, povidone, silicon dioxide, colloidal silicon dioxide, silicone, silicone adhesive 4102, and silicone emulsion. It should be understood, however, that the carriers selected for the pharmaceutical compositions, and the amounts of such carriers in the composition, may vary depending on the method of formulation (e.g., dry granulation formulation, solid dispersion formulation).

Pharmaceutically acceptable carrier: Any ingredient other than the disclosed PARP-1 inhibiting compounds, or a pharmaceutically acceptable salt thereof (e.g., a carrier capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

All ranges disclosed and/or claimed herein are inclusive of the recited endpoint and independently combinable. For example, the ranges of “from 2 to 10” and “2-10” are inclusive of the endpoints, 2 and 10, and all the intermediate values between in context of the units considered. For instance, reference to “Claims 2-10” or “C₂-C₁₀ alkyl” includes units 2, 3, 4, 5, 6, 7, 8, 9, and 10, as claims and atoms are numbered in sequential numbers without fractions or decimal points, unless described in the context of an average number. The context of “pH of from 5-9” or “a temperature of from 5° C. to 9° C.”, on the other hand, includes whole numbers 5, 6, 7, 8, and 9, as well as all fractional or decimal units in between, such as 6.5 and 8.24.

It is also understood that for all descriptions herein referring to a compound of Formula (I), or a pharmaceutically acceptable salt, co-crystal, ester, solvate, hydrate, isomer (including optical isomers, racemates, or other mixtures thereof), tautomer, isotope, or polymorph thereof, or a subgeneric embodiment thereof, there are such descriptions for each embodiment of a compound of Formula (II), Formula (III), Formula (IV), Formula (VI), Formula (VII), and all subgeneric embodiments for each described herein. For instance, for each pharmaceutical composition and method of treatment or use described herein utilizing a compound of Formula (I), additional such pharmaceutical compositions and methods of treatment or use are included for each embodiment of a compound of Formula (II), Formula (III), Formula (IV), Formula (VI), Formula (VII), and all subgeneric embodiments for each described herein.

Poly(ADP-ribose) polymerase 1 (PARP-1)—the founding member of a large enzyme family (16 active members)—is a multidomain enzyme that catalyzes the post-translational modification known as ADP-ribosylation (PARylation) using nicotinamide adenine dinucleotide (NAD⁺) as a substrate (1). PARP-1 plays critical roles in transcriptional regulation, DNA repair, and replication fork protection during S phase (2). Cancer cells that have defects in DNA repair or have high levels of replication stress are uniquely sensitive to PARP-1 inhibition by NAD⁺-competitive small molecules inhibitors (3, 4). Currently, there are four (olaparib, rucaparib, niraparib, and talazoparib) FDA-approved PARP-1 inhibitors and several others in clinical development (e.g., veliparib).

Intriguingly, the cancer cell cytotoxicity of clinical PARP-1 inhibitors does not corelate with the degree of cellular PARylation inhibition (2). Beyond catalytic inhibition, some PARP-1 inhibitors “trap” PARP-1 on chromatin in response to DNA damaging agents (5). This trapped PARP-1 inhibitor—DNA complex is hypothesized to be the driver of cytotoxicity (6). While the concept of PARP-1 trapping has gained popularity, its molecular nature is poorly understood.

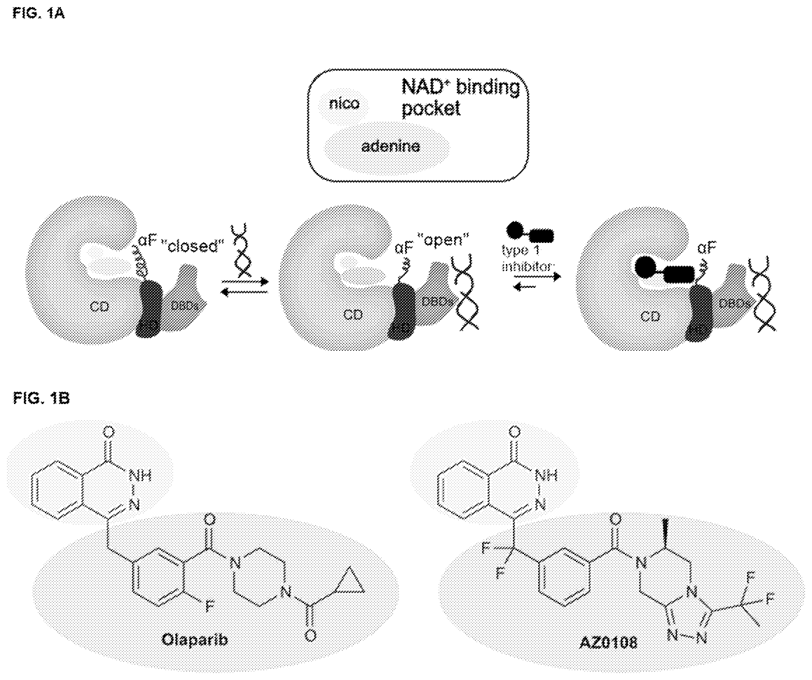

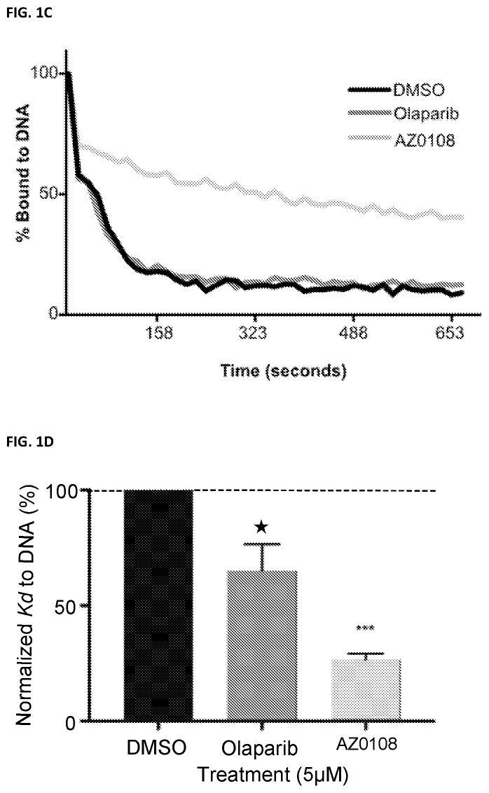

PARP-1 mediated PARylation is tightly regulated by long-range allosteric coupling between the NAD⁺-binding pocket and the DNA binding domain (7, 8). Under basal conditions, the adenine sub-pocket of the NAD⁺ binding pocket is sterically occluded by an alpha helix (αF) in the helical domain, preventing the binding of NAD⁺ (FIG. 1A) (9). When PARP-1 binds to DNA breaks, αF partially unfolds due to interdomain interactions between the DNA binding domains and the helical domain. The unfolding of αF relieves the steric block on the adenine sub-pocket, allowing NAD⁺ to bind. Thus, the catalytic activity of PARP-1 is regulated by allosteric control of substrate binding.

Allosteric regulation of PARP-1 can also occur in the “reverse” direction: from the NAD⁺ binding pocket to the DNA binding sites. The non-hydrolyzable NAD⁺ analog benzamide adenine dinucleotide (BAD) destabilizes αF, leading to an increase in the affinity of PARP-1 for damaged DNA (FIG. 1A) (10). Benzamide, which occupies only the nicotinamide sub-pocket, does not increase the affinity of PARP-1 for DNA breaks. These results show that the adenine sub-pocket—αF interface plays a critical role in allosteric coupling between the NAD⁺ binding pocket and the DNA binding sites.

While all clinical PARP-1 inhibitors bind in the nicotinamide sub-pocket, their degree of engagement with the adenine sub-pocket—αF interface varies substantially. One model posits that inhibitor-induced PARP-1 trapping correlates with the ability of PARP-1 inhibitors to cause allosteric retention of PARP-1 onto DNA breaks, like BAD. A recent study sought to test this model by evaluating the effects of various PARP-1 inhibitors on DNA binding in vitro (11). This resulted in the classification of PARP-1 inhibitors based on their impact on PARP-1 binding to DNA: i. allosteric pro-retention inhibitors (Type I inhibitors) (FIG. 1A), ii. mild or no-allosteric retention inhibitors (Type II inhibitors), and iii. allosteric pro-release inhibitors (Type III inhibitors). Surprisingly, none of the clinical PARP-1 inhibitors are Type I inhibitors; in fact, the only type I inhibitor identified was EB-47, a structural mimic of NAD⁺ that is a pan-PARP inhibitor. These results suggest that PARP-1 trapping by clinical inhibitors is not due to inhibitor-mediated allosteric retention of PARP-1 onto damaged DNA. Nevertheless, conversion of a type III inhibitor (veliparib) into a type I inhibitor (UKTT15) resulted in increased cytoxicity in cancer cells deficient in DNA repair. Thus, type I inhibitors have therapeutic potential for cancer treatment. However, several critical questions remain: What are the key molecular features that give rise to type I inhibition? Can type I inhibition be tuned by differential engagement of adenine sub-pocket—αF interface? Are type I inhibitors mechanistically distinct from type II inhibitors in terms of downstream cellular consequences of PARP-1 inhibition?

Herein, we show that AZ0108, a phthalazinone triazolo[4,3-a]pyrazines-based PARP-1 inhibitor (12), is a type I inhibitor. AZ0108 has the same core scaffold as olaparib (a type II inhibitor), but has different substituents emanating from the C-4 position that occupies the adenine sub-pocket. Unlike olaparib, AZ0108 induces replication stress and S phase arrest. The AZ0108-induced allosteric pro-retention is dependent on specific interactions within adenine sub-pocket-αF interface. Synthesis of a series of phthalazinone triazolo[4,3-a]pyrazines (Pips) show that the degree of allosteric pro-retention can be tuned. Surprisingly, we found that an isosteric analogue of AZ0108 (i.e., Pip6), which is equipotent to AZ0108 in terms of in vitro PARylation inhibition and replication stress induction, exhibited an ˜80-fold increase in cytotoxicity in Ewing Sarcoma cells. The increased cytotoxicity of Pip6 relative to AZ0108 is due to an increase in cellular residence time on PARP-1. Thus, the cytotoxicity of phthalazinone-based, type I inhibitors is driven not only by replication stress induction, but also increased cellular target residence time.

### Results

### AZ0108 is a Type I Inhibitor of PARP-1

The adenine sub-pocket-αF interface plays a key role in regulating inhibitor-induced retention of PARP-1 on DNA breaks (10). PARP-1 inhibitors can engage the adenine sub-pocket, but it is clear that occupancy alone is not sufficient to drive retention of PARP-1 on DNA breaks. We hypothesized that if an inhibitor engages the adenine sub-pocket in a similar manner to the adenine of BAD, that it would drive retention of PARP-1 on DNA breaks. To test this hypothesis, we sought to compare the DNA binding effects of inhibitors that make similar contacts in the nicotinamide sub-pocket, but differ in the substituents that occupy the adenine sub-pocket. We compared two structurally related phthalazinone-based inhibitors, olaparib (a type II inhibitor) and AZ0108 (12) (FIG. 1B). These inhibitors share the same core phthalazinone scaffold, but differ greatly in the C-4 piperazine substituent that engages the adenine sub-pocket: olaparib contains a 4-cyclopropanecarbonyl piperazine whereas AZ0108 contains a triazolo[4,3-a]pyrazine with a S-methyl at the C-6 position of the piperazine ring and a difluoroethyl group at the C-3 position of the triazole ring (FIG. 1B). AZ0108 and olaparib are most potent against PARP-1 and the closely related family member PARP-2; however, AZ0108 also potently inhibits PARP-6, which catalyzes mono-ADP-ribosylation (MARylation) rather than PARylation.

Using in vitro PARylation assays, we confirmed that AZ0108 and olaparib have similar potencies against PARP-1 catalytic activity (IC₅₀=12 nM versus 19 nM). To evaluate the effects of AZ0108 and olaparib on the binding of PARP-1 to nicked DNA, we used an in vitro fluorescence polarization DNA competition assay that we previously described (10, 11). We found that AZ0108 substantially reduced the exchange of the PARP-1:labled DNA complex with an unlabeled DNA competitor (FIG. 1C). In contrast, olaparib did not impact this exchange (FIG. 1C), which is consistent with olaparib being a type II inhibitor. AZ0108 increased the affinity of PARP-1 by 4-fold whereas olaparib only had a modest effect (FIG. 1D). These results demonstrate that AZ0108 is an allosteric pro-retention (type I) inhibitor, and show that the nature of substituent at the C-4 on the phthalazinone scaffolds determines inhibitor type.

### Type I Inhibition of PARP-1 by AZ0108 Induces Pan-Nuclear γH2AX

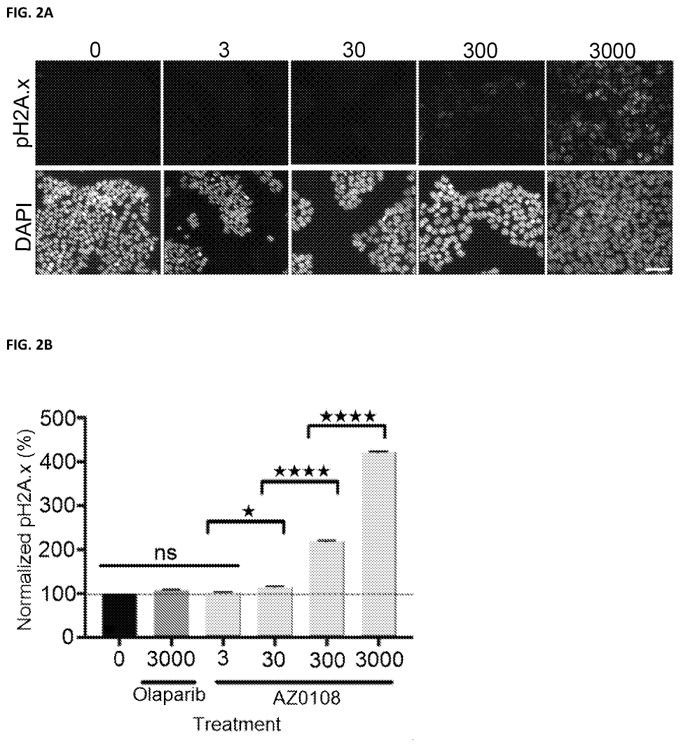

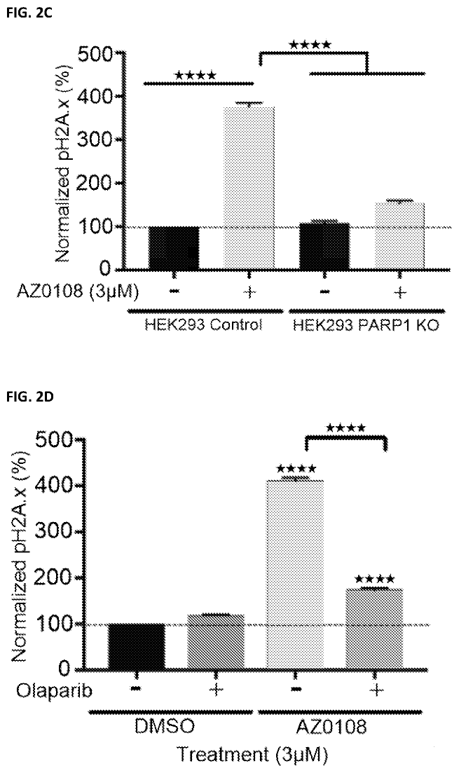

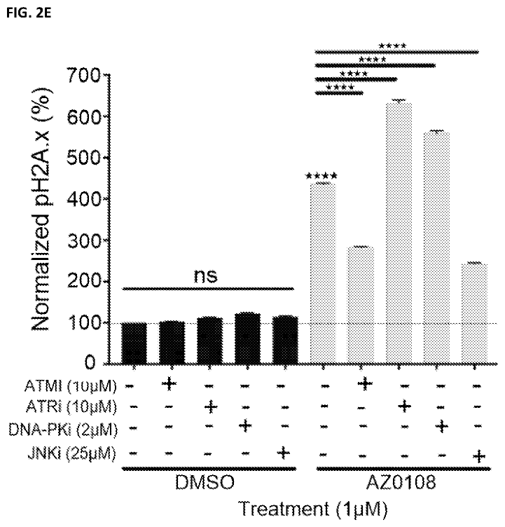

Given our finding that AZ0108 promotes retention of PARP-1 on DNA breaks in vitro, we wondered what impact this would have on PARP-1 signaling in unperturbed cells. Surprisingly, we found that treatment of human embryonic kidney (HEK) 293 cells with AZ0108 resulted in a dose-dependent increase in pan-nuclear H2AX phosphorylation at Ser139 (referred to as gamma-H2AX, γH2AX) (FIGS. 2A, 2B). Pan-nuclear γH2AX is associated with replication stress, whereas γH2AX foci is a marker of DNA damage (13). Olaparib did not induce either pan-nuclear γH2AX or γH2AX foci. To further confirm the lack of DNA damage induction by AZ0108 we performed the neutral comet assay to assess double-strand breaks (DSBs) (14). Compared to the known DNA damage inducer, bleomycin, AZ0108 only weakly generated DSBs in HEK 293T cells. Thus, AZ0108-induced pan-nuclear γH2AX is associated with induction of replication stress and not DSB formation.

PARP-1 is required for the ability of type II PARP-1 inhibitors to kill cancer cells defective in DNA repair (6). We therefore determined if the effects of AZ0108 on pan-nuclear γH2AX is dependent on PARP-1. Using PARP-1 knockout (KO) 293 cells, we found that the AZ0108-induced pan-nuclear γH2AX signal was dependent on the presence of PARP-1, suggesting that the AZ0108-PARP-1 complex promotes replication stress (FIG. 2C). Consistent with this notion, co-treatment with olaparib, which competes with AZ0108 for binding to PARP-1, reversed the effects of AZ0108 on pan-nuclear γH2AX (FIG. 2D). Together, these results support the notion that the binding of a type I inhibitor to PARP-1 can drive replication stress in unperturbed cells.

Next, we investigated which kinases phosphorylate H2AX in response to AZ0108. There are three major H2AX kinases, namely ataxia-telangiectasia-mutated (ATM) and ATR (15). We found that inhibition of ATM by KU-55933 partially reduced (˜2-fold) AZ0108-induced pan-nuclear γH2AX (FIG. 2E). This partial reduction in pan-nuclear γH2AX levels motivated us to search for other potential H2AX kinases that act downstream of AZ0108-induced replication stress. H2AX is also a target of ATR and DNA-PK (15); however, inhibition of ATR by VE-821 or DNA-PK by NU7441 slightly increased (˜1.3 fold) AZ0108-induced pan-nuclear γH2AX (FIG. 2E). In addition to the canonical H2AX kinases described above, c-Jun N-terminal kinase (JNK) can phosphorylate H2AX in the presence of UV-induced replication stress (16). Indeed, we found that inhibition of JNK by SP600125 partially reduced (˜2-fold) AZ0108-induced pan-nuclear γH2AX (FIG. 2E). Taken together, these results show that both ATM and JNK phosphorylate H2AX in response to AZ0108-PARP-1 complex-induced replication stress. Additionally, inhibition of the catalytic activity of ATR and DNA-PK exacerbates the effects of AZ0108-induced pan-nuclear γH2AX.

Replication stress triggered by replication inhibitors activates the S phase check point, leading to the recruitment and activation of ataxia telangiectasia-mutated and Rad3-related (ATR) kinase and its downstream target Chk1 (17). ATR-mediated phosphorylation of Chk1 at Ser345 leads to its activation, which is critical for mediating cellular responses to replication stress. Consistent with previous studies in breast cancer cells (18), treatment of HEK 293 cells with AZ0108 induced Ser345 phosphorylation of Chk1. In breast cancer cells, this effect was attributed to AZ0108-mediated inhibition of PARP-6 catalytic activity, and not PARP-1 (18). However, we found that full-length PARP-6, which contains the intact catalytic domain, is not present in HEK 293T cells. Hence, we attribute AZ0108-induced Ser345 phosphorylation of Chk1 to AZ0108-mediated Type I inhibition of PARP-1. AZ0108-induced Ser345 phosphorylation of Chk1 was significantly inhibited by the ATR inhibitor VE-821. This is consistent with the notion that the AZ0108-PARP-1 complex induces replication stress, which leads to activation of ATR and its downstream targets.

### Type I Inhibition of PARP-1 by AZ0108 Induces S-Phase Arrest and Senescence in HEK 293 Cells

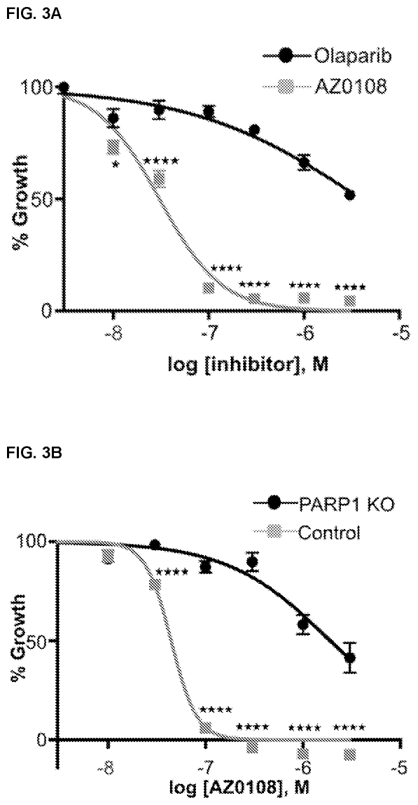

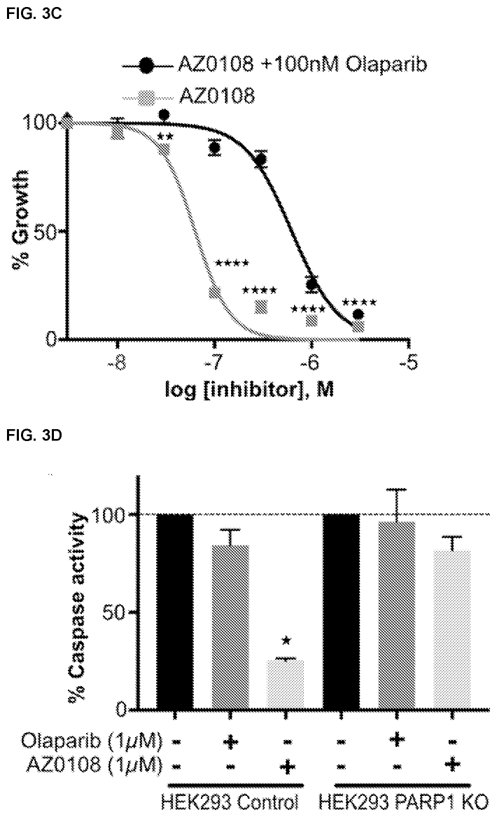

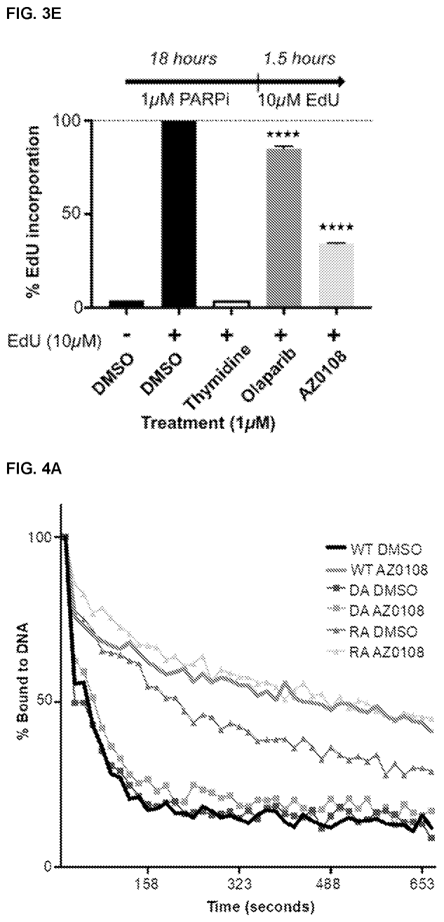

Activation of the ATR-CHK1 signaling pathway leads to cell cycle arrest (17). Using the MTT assay, we found that AZ0108 inhibited cell growth (72 h) in a dose dependent manner (EC₅₀=30 nM) (FIG. 3A). Olaparib had little effect on cell viability (EC₅₀=˜10 μM) (FIG. 3A). KO of PARP-1 resulted in a 43-fold increase in the EC₅₀ value for AZ0108, demonstrating that PARP-1 is required for the effects of AZ0108 on cell growth (FIG. 3B). When dual-treated, olaparib right-shifted the EC₅₀ value for AZ0108 on cell growth by 10-fold, presumably by competing for binding to PARP-1 (FIG. 3C). To determine whether the impairment in viability in HEK 293 cells after AZ0108 treatment is due to apoptosis, we used a caspase-3/7 activity assay. AZ0108 reduced caspase-3/7 activity by 75%; by contrast, olaparib did not significantly affect caspase-3/7 activity (FIG. 3D). The effects of AZ0108 on caspase-3/7 activity were dependent on PARP-1 (FIG. 3D). These results support the notion that AZ0108-PARP-1 complex formation induces senescence and not apoptosis in HEK 293 cells.

We next determined the stage of cell cycle-arrest by AZ0108 in HEK 293 cells. By using DAPI fluorescence, cell counts were mapped to cell cycle using FACS analysis. Compared to thymidine, a known inhibitor of the S-phase, AZ0108 also arrested HEK 293 cells in S-phase. Olaparib on the other hand did not alter the cell cycle and displayed similar cell cycle counts compared to DMSO.

### DAPI Intensity

| | G1 | S | G2 |
|---|---|---|---|
| DMSO | 42.2% | 44.1% | 9.06% |
| Thymidine (2 mM) | 2.79% | 76.3% | 7.06% |
| Olaparib (1 μM) | 19.4% | 54.0% | 9.50% |
| AZ0108 (1 μM) | 9.31% | 77.4% | 9.65% |

We also used the thymidine analog 5-ethynyl-2′-deoxyuridne (EdU) followed by the copper-mediated Huisgen cycloaddition reaction (“click chemistry”) to monitor cells in S-phase (19). AZ0108 blocked EdU incorporation by 65%; by contrast, olaparib minimally blocked EdU incorporation. Taken together, these results show that type I inhibition of PARP-1 leads to S phase arrest and cell senescence in HEK 293 cells.

### AZ0108 Mediates Reverse Allostery by Disrupting Interdomain Contacts Between the Helical Domain and the Catalytic Domain

We next sought to gain molecular insight into how AZ0108 acts as a type I inhibitor of PARP-1 (i.e., how AZ0108 stabilizes PARP-1 binding to DNA via reverse allostery). We generated a model of AZ0108 bound to PARP-1 using an induced fit docking (IFD) protocol (Schrödinger). The IFD top-ranked pose for AZ0108 had a docking score of 13.43 kcal/mol, which is energetically favorable. The phthalazinone core occupies the nicotinamide binding sub-pocket, whereas the triazolo[4,3-a]pyrazine substituent of AZ0108 occupies the adenine sub-pocket. The C-3 1,1-difluoroethyl group attached to the triazolo[4,3-a]pyrazine substituent abuts Asp770, which is located at the C-terminus of αF in the helical domain. Asp770 is one of the key amino acids that mediates allosteric coupling between the DNA binding domain with the catalytic domain (10, 11), perhaps via its interaction with Arg878. This is interaction could potentially disrupt the inter-domain salt bridge between Asp770 and Arg878, which is located on the D-loop in the catalytic domain.

To further explore the effects of AZ0108 on the interactions between the catalytic domain and the helical domain we performed molecular dynamic (MD) simulations. During the 200 ns MD simulations, the helical domain moved away from the catalytic domain upon binding AZ0108. This was quantified by measuring the residue-residue distance distribution for Arg878(Cα)-Asp770(Cα) and Tyr889(Cα)-Val758(Cα). We did not observe inter-domain movement in the MD simulation of the apo PARP-1 structure. The MD simulation of the AZ0108 bound structure supports the notion that the inter-domain salt bridge interaction between R878 and D770 is destabilized when AZ0108 occupies the catalytic site of PARP-1.

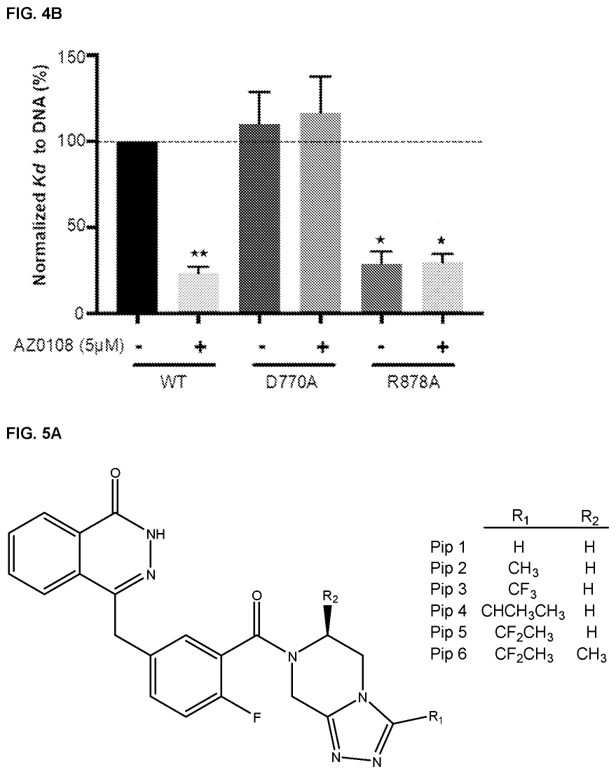

In order to validate our modeling and MD simulation experiments, mutagenesis studies were performed. We generated two mutants of PARP-1, Asp770 to alanine (D770A) and Arg878 to alanine (R878A), and determined the effects of AZ0108 on the binding of PARP-1 to nicked DNA using the in vitro fluorescence polarization DNA competition assay. Strikingly, AZ0108 did not impact the exchange of the D770A PARP-1:labled DNA with unlabeled DNA nor did it change D770A PARP-1's affinity for DNA, unlike what we observed for WT PARP-1 (FIG. 4A, 4B). DNA binding to R878A PARP-1 was similarly unaffected by AZ0108; however, R878A PARP-1:labled DNA showed reduced exchange with unlabeled DNA and R878A PARP-1 had an increased affinity for DNA compared to WT PARP-1 (FIG. 4A, FIG. 4B). This increase in DNA binding affinity for R878A PARP-1 relative to WT PARP-1, in the absence of AZ0108, reveals the importance of Arg878 in allosteric coupling between the DNA binding domain with the catalytic domain. One possible reason for the lack of effect of AZ0108 on DNA binding to either the D770A or R878A PARP-1 mutants is that AZ0108 doesn't bind to these PARP-1 mutants. To test this possibility, we expressed WT, D770A, and R878A PARP-1 in PARP-1 KO HEK 293 cells. Similar to WT PARP-1, both D770A and R878A PARP-1 were inhibited by AZ0108, suggesting that AZ0108 binding is not perturbed in the PARP-1 mutants. Intriguingly the D770A mutant was ˜2-fold more active than WT PARP-1 whereas R878A was ˜10-fold less active. Further studies are required to understand the alterations in activity of the mutants, but it is likely due to perturbations of the allosteric coupling between the catalytic domain and the DNA binding domain. Taken together, our results support the notion that AZ0108 acts as a type I inhibitor by perturbing the interdomain salt bridge between Arg878 and Asp770.

A Series of Phthalazinone Triazolo[4,3-a]Pyrazines Reveals that Type I Inhibition can be Finely Tuned

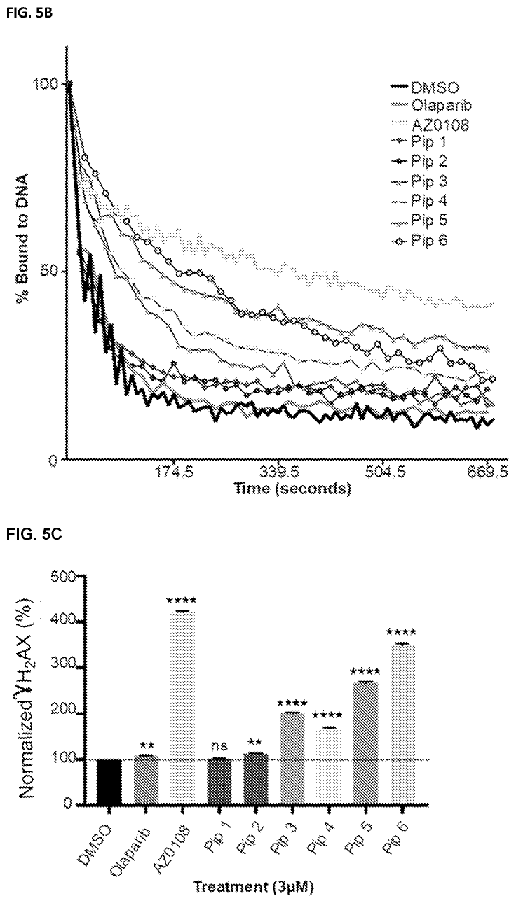

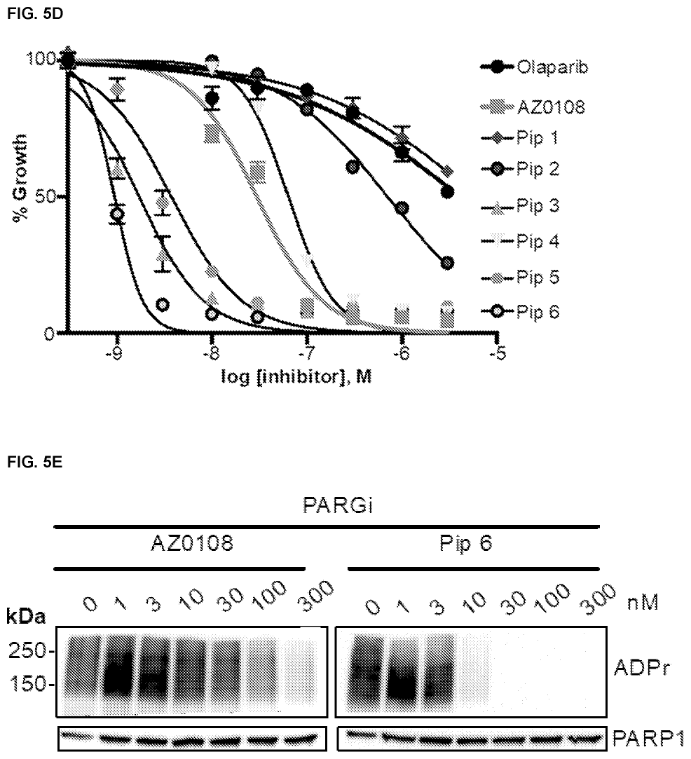

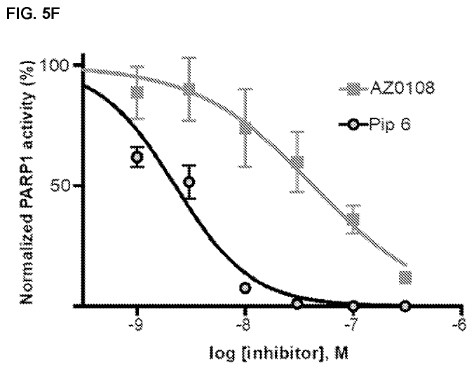

We next focused on understanding what structural features of AZ0108 contribute to type I inhibition of PARP-1. The molecular modeling and MD simulations suggested that the C-3 1,1-difluoroethyl group attached to the triazolo[4,3-a]pyrazine substituent of AZ0108 is a key feature contributing to type I inhibition of PARP-1. We therefore synthesized a series of phthalazinone triazolo[4,3-a]pyrazines (Pips) that have various alkyl and fluoroalkyl substituents at the C-3 position of the triazolo[4,3-a]pyrazine substituent (Pip1-5) (FIG. 5A). Pip3 was previously described (20). We also synthesized a close isostere of AZ0108 that contains an S-methyl group at the C-6 position of the piperazine ring (Pip6) (FIG. 5A). An important feature to point out is that the Pip series differs from AZ0108 in that they contain a methylene group rather than a difluoro group at the benzyl linker at the C-4 position of the phthalazinone scaffold (FIG. 5A).

To confirm that these Pip compounds inhibit PARP-1 catalytic activity, we assessed PARP-1 mediated auto-PARylation via an in vitro assay (21, 22). All Pips potently inhibited PARP-1 auto-PARylation (IC₅₀ ˜4-17 nM). The most potent inhibitor, Pip1 (IC₅₀=3.9 nM), contains a hydrogen at the C-3 position. Increasing the steric bulk at the C-3 position generally reduced potency; the least potent inhibitor (IC₅₀=17.5 nM) was Pip4, which contains an isopropyl at the C-3 position. Pip6 and AZ0108 are equipotent suggesting that the difluoro group at the C-4 position of the phthalazinone scaffold does not contribute to inhibitor potency.

We next examined the effects of Pip1-6 on the binding of PARP-1 to nicked DNA using the in vitro fluorescence polarization DNA competition assay. Pip3-6 reduced, to varying degrees, the exchange of the PARP-1:labled DNA with an unlabeled DNA competitor and increased the affinity of PARP-1 for nicked DNA compared to the DMSO control (FIG. 5B). By contrast, Pip1,2 had very little impact on the exchange of the PARP-1:labled DNA with an unlabeled DNA competitor and had similar effects to olaparib on the affinity of PARP-1 for nicked DNA (FIG. 5B). Thus, the identity of C-3 group can impact type I inhibition of PARP-1, with larger alkyl and fluoroalkyl groups having the most profound effect on reverse allostery. Compared to Pip6, AZ0108 has a more pronounced effect on DNA binding to PARP-1; this demonstrates that while the difluoro group at the C-4 position doesn't affect PARP-1 auto-PARylation inhibition potency, it contributes to the reverse allosteric effect of AZ0108. The finding that Pip6 has a more pronounced effect on DNA binding than Pip5 shows that the S-methyl group at the C-6 position of the piperazine ring also contributes to type I inhibition.

Having demonstrated that some Pips are type I inhibitors, we next examined their effects on pan-nuclear γH2AX. The magnitude of pan-nuclear γH2AX induction was as follows: AZ0108>Pip6>Pip5>Pip3˜ Pip4>>Pip2˜ Pip1˜ olaparib (FIG. 5C). These results generally correlate with the effects of the Pips on in vitro DNA binding described above, suggesting that the degree of type I inhibition generally correlates with extent of replication stress induction. The cytotoxicity of Pip6 is driven by target residence time in cells and type I inhibition

Next, we examined the effects of Pips on HEK 293 cell growth using the MTT assay. Unexpectedly, several of the Pips (namely, Pip3, Pip5, and Pip6) were substantially more potent than AZ0108 in inhibiting cell growth (FIG. 5D). This was surprising given the fact that Pip3, Pip5, and Pip6 induced pan-nuclear γH2AX to a lesser extent than AZ0108. The most potent Pip was Pip6, which was 30-fold more potent than AZ0108 (EC₅₀=0.94 nM versus 29.7 nM). Pip1, which is not a type I inhibitor, was the least potent, with an EC₅₀ ˜6 μM (FIG. 5D). This is intriguing because Pip1 is more potent than Pip6 in inhibiting PARP-1 catalytic activity in vitro. We compared the activity of Pip6 and AZ0108 against PARP-1-mediated PARylation in HEK 293 cells. To visualize PARylation in cells more readily, we blocked PAR-removal using an inhibitor (i.e., PDD00017273) of poly-ADP-ribose glycohydrolase (PARG) (23). Therefore, the PARylation signal after brief (15 min) PDD00017273 treatment is due to PARP-1 activity alone. While Pip6 and AZ0108 exhibit similar potency against PARP-1 auto-PARylation in vitro, Pip6 is ˜20-fold more potent than AZ0108 in inhibiting PARP-1 PARylation in HEK 293 cells (EC₅₀=2.3 nM versus 42.5 nM) (FIG. 5E). Collectively these results are intriguing and suggest that in addition to type I inhibition, other properties of Pip6 (and perhaps Pip3 and Pip5)—independent of in vitro potency against PARP-1 catalytic activity—contribute to potent cell growth inhibition.

Based on our results above, we hypothesized that the increased cellular potency of Pip6 compared to AZ0108, is due to increased target residence time. To test this hypothesis, we performed in-cell competition labeling experiments using an olaparib-based clickable photoaffinity labeling (PAL) probe (ad-olpaparib) (24). ad-olaparib contains a “minimalist” linker containing a diazirine and a terminal alkyne, below.

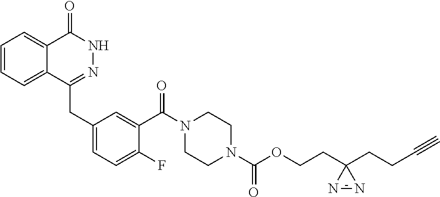 [SMILES: C#CCCC1(CCOC(=O)N2CCN(C(=O)c3cc(Cc4n[nH]c(=O)c5ccccc45)ccc3F)CC2)N=N1]

Treatment of HEK 293 cells with ad-olaparib followed by UV irradiation (350 nm) and copper-catalyzed conjugation to a TAMRA-azide, results in covalent labeling of a band corresponding to the molecular weight of endogenous PARP-1, which can be detected by in-gel fluorescence (FIG. 6A). Using PARP-1 KO cells, we confirmed that this band was indeed PARP-1. Pre-treatment with either Pip6 or AZ0108 substantially blocked the labeling of PARP-1 by ad-olaparib. Even after washout (3 h), the labeling of PARP-1 by ad-olaparib was not recovered in Pip6 treated cells; by contrast, the labeling of PARP-1 by ad-olaparib was recovered in AZ0108 treated cells more than 2-fold. Similar results were obtained by performing in vitro competition labeling experiments with recombinant His6-PARP-1. Together, these results suggest that Pip6 has a longer residence time on PARP-1 (in cells and in vitro) compared to AZ0108.

To corroborate our in-cell competition labeling experiments, we analyzed the effects of Pip6 and AZ0108 on PARP-1-mediated PARylation activity after extensive washout. HEK 293 cells were treated with Pip6 or AZ0108 followed by washout for increasing times. Cells were then treated with a PARG inhibitor (PDD00017273) for 15 min. Prior to washout, both Pip6 and AZ0108 inhibited PARP-1-mediated PARylation activity by more than 90%. In AZ0108 treated cells, PARP-1 mediated PARylation completely recovered after 3 h of washout. Strikingly, in Pip6 treated cells, PARP-1 mediated PARylation only partially recovered (˜10%), even after 12 h of washout. These data are consistent with the competition experiments using ad-olaparib and support the notion that the increased cellular activity of Pip6 compared to AZ0108, is due to increased residence time on PARP-1.

### Pip6 Exhibits Sub-Nanomolar Cytotoxicity in Ewing Sarcoma EW-8 Cells

Having demonstrated that Pip6 is a type I inhibitor with a long residence time, we wanted to compare its efficacy to AZ0108—and to other clinically relevant PARP-1 inhibitors—in killing cancer cells. We focused on Ewing sarcoma, which is the second most common type of bone cancer in children (25). Despite no defects in DNA repair pathways (26), several Ewing sarcoma cell lines are sensitive to PARP-1 inhibitors. Pip6 potently inhibited (EC₅₀=0.24 nM) the viability of the Ewing sarcoma cell line EW-8. Pip6 was ˜3-fold more potent than SN-38, a topoisomerase inhibitor currently used to treat the Ewing sarcoma (25); and was 59-fold more potent than talazoparib (type II inhibitor), which is the most potent clinical PARP inhibitor. AZ0108 also inhibited (EC₅₀=20 nM) the viability of EW-8 cells, but was 86-fold less potent than Pip6. Taken together, these results show that the increased PARP-1 residence time for Pip6 compared to AZ0108, translates to increased cytoxicity in Ewing sarcoma cells.

| | EC50 (nM) | Fold Difference to Pip6 |
|---|---|---|
| Pip6 | 0.24 | 1 |
| SN-38 | 0.75 | 3 |
| Talazoparib | 14.20 | 59 |
| AZ0108 | 20.92 | 86 |
| Olaparib | 1112.06 | 4590 |
| Niraparib | 1379.36 | 5693 |
| Rucaparib | 5572.36 | 22998 |
| Veliparib | 59562.25 | 245827 |

### Discussion

While all PARP-1 inhibitors are NAD⁺ competitive and thus inhibit PARP-1 mediated PARylation, they vary widely in terms of their influence on PARP-1 binding to DNA. In this study, we show that the phthalazinone triazolo[4,3-a]pyrazine PARP-1 inhibitor, AZ0108, induces PARP-1 retention on DNA via reverse allostery (referred to as a type I inhibitor), leading to replication stress, S phase arrest, and cell senescence in HEK 293 cells. The synthesis of six phthalazinone triazolo[4,3-a]pyrazines (Pips) related to AZ0108 revealed that subtle changes to the substituent emanating from C-4 have profound impacts on type I inhibition and the extent of replication stress induction. Unexpectedly, an isosteric analogue of AZ0108, Pip6, showed equivalent activity against PARP-1 catalytic activity in vitro and a similar magnitude of replication stress induction, yet was ˜100-fold more potent in inhibiting cell growth. Washout and in-cell competition labeling experiments revealed that Pip6 has a much longer target residence time on PARP-1 compared to AZ0108. This translated to an enhanced cytotoxicity of Pip6 compared to AZ0108 in Ewing Sarcoma cells. Taken together, our results provide molecular and cellular insights into type I inhibition of PARP-1 by phthalazinone triazolo[4,3-a]pyrazines and suggest strategies for enhancing the cancer cell cytoxicity of type I inhibitors of PARP-1.

AZ0108 and the related Pips described in our study join a small, but growing list of type I inhibitors of PARP-1; these include the NAD⁺ mimetics, BAD and EB-47, and the veliparib-related analog, UKTT15 (10, 11). Hydrogen/deuterium exchange mass spectrometry (HXMS) show that these type I inhibitors increase the affinity of PARP-1 for DNA by destabilizing αF of the helical domain (11). However, structural studies of EB-47 and UKTT15 bound to the catalytic domain of PARP-1 reveal that EB-47 and UKTT15 do so by different mechanisms located at different positions along αF: the extended phenyl group at the C-2 position of UKTT15 clashes with amino acids in the N-terminal region of αF, whereas EB-47 disrupts contacts between the adenine sub-pocket—αF interface in the middle region of αF. Consistent with this notion, mutation of Asp766 and Asp770, located in the middle region of αF, conferred resistance of type I inhibition by EB-47 whereas these mutations had no effects on type I inhibition by UKTT15 (11). Comparison of the structure of EB-47:PARP-1 to the structures of olaparib:PARP-1 and apo PARP-1 reveals that the adenine of EB-47 displaces the D-loop Arg878 in the catalytic domain relative to its position in the olaparib:PARP-1 or apo PARP-1 structures. Displacement of Arg878 disrupts the salt bridge between Arg878 and Asp770 (located in the middle of αF) observed in the olaparib:PARP-1 and apo PARP-1. While we do not have a structure of AZ0108 bound to PARP-1, our modeling and molecular dynamic simulations, support the idea that the C-3 difluoroethyl substituent on the triazolo[4,3-a]pyrazine group of AZ0108 similarly displaces Arg878, and by doing so, disrupts the Arg878-Asp770 salt bridge. Our finding that mutation of either Asp770 or Arg878 eliminates the effects of AZ0108 on PARP-1 binding to DNA supports this hypothesis. These results also establish the importance of the Arg878-Asp770 salt bridge at the adenine sub-pocket—αF interface as a key interaction mediating long-range allosteric coupling between the catalytic domain and the DNA binding domain of PARP-1.

AZ0108, but not olaparib, induces pan-nuclear γH2AX (associated with replication stress), S phase arrest, and cellular senescence in HEK 293 cells, demonstrating that these cellular phenotypes are specifically mediated by type I inhibition of PARP-1. Knockout of PARP-1 demonstrated that these cellular phenotypes are driven by the AZ0108-PARP-1 complex. How might the AZ0108-PARP-1 complex cause replication stress? In unperturbed cells, PARP-1 is activated during DNA replication in S phase by unligated Okazaki fragments (27). AZ0108 binding to PARP-1 could slow down or prevent the release of PARP-1 from unligated Okazaki fragments at the replication fork via type I inhibition of PARP-1. If AZ0108 prevents the release of PARP-1 from the replication fork this could lead to replication stress, S phase check point activation and cellular senescence. It will be interesting in future studies to determine if replication stress induced by type I inhibition of PARP-1 is mechanistically distinct from other types of replication-stress inducing agents (e.g. topoisomerase inhibitors).

We are intrigued by our data showing that Pip6 has a longer residence time on PARP-1 compared to AZ0108. As shown in FIG. 5, the only difference between Pip6 and AZ0108 is the identity of benzyl linker emanating from the C-4 position of the phthalazinone scaffold: in Pip6 it is a methylene whereas in AZ0108 it is a difluoromethylene. How do these seemingly subtle differences result in dramatic differences in inhibitor residence time on PARP-1? The methylene in benzyl linker of Pip6 is expected to have a tetrahedral geometry (i.e, sp³ hybridization) whereas the difluoromethylene in benzyl linker of AZ0108 is expected to have a more trigonal planar geometry (i.e, sp² hybridization) (28). We speculate that the geometry of the benzyl linker will impact how the triazolo[4,3-a]pyrazine substituent is oriented in the adenine sub-pocket of the NAD⁺ binding site, thereby influencing inhibitor dissociation rates. Another possibility is that alteration of the H-bond acceptor (HBA) strength of the carbonyl in the phthalazinone scaffold impacts residence time on PARP-1. Using quantum mechanical electrostatic potentials, a previous study found that a difluoromethylene at C-4 position in a closely related phthalazinone triazolo[4,3-a]pyrazine PARP-1 inhibitor increased the HBA strength of the carbonyl in the phthalazinone scaffold compared to a methylene at the C-4 position (20). Ultimately, solving the structures Pip6 and AZ0108 bound to PARP-1 will provide molecular insights into the differences in inhibitor residence time on PARP-1.

The prolonged PARP-1 residence time of Pip6 compared to AZ0108 conferred enhanced cytotoxicity in the Ewing sarcoma cell line, EW-8. Pip6, which exhibited sub-nanomolar cytotoxicity in EW-8, was substantially more potent than all clinically relevant PARP-1 inhibitors; the order of cytoxicity is as follows: Pip6>>>talazoparib ˜AZ0108>>olaparib ˜ niraparib >rucaparib >>veliparib. The cytotoxicity of PARP-1 inhibitors as single agents in EW-8 cells does not correlate with their potency against PARP-1 catalytic activity, cellular PARP-1 trapping (i.e., chromatin enrichment of PARP-1) ability in unperturbed cells (29), or their type of PARP-1 inhibition (i.e., reverse allosteric effects on DNA binding). What, then explains the differences in cytotoxicity among clinically relevant PARP-1 inhibitors? While several factors likely contribute, our data comparing Pip6 and AZ0108 support the notion that PARP-1 target residence time is a major contributor to PARP-1 inhibitor cytoxicity. Other possibilities include polypharmacology (i.e. targeting multiple PARP family members) and influences on PARP-1 binding to essential regulatory factors (e.g., histone PARylation factor 1, HPF1) that regulate PARP-1 activity in cells. In the case of Pip6, we believe that potent cytoxicity is achieved by a combination of type I inhibition and long PARP-1 residence time in cells and not PARP polypharmacology. It will be interesting in future studies to determine if Pip6, unlike talazoparib and olaparib, is effective as a single agent in improving survival in a mouse model of Ewing sarcoma.

Overall, our study not only provides deeper mechanistic insight into type I inhibition of PARP-1, but also potential strategies for improving the cancer cell cytoxicity of PARP-1 inhibitors.

### Methods

### Fluorescence Polarization Assay

The DNA competition fluorescence polarization assay was performed as described (Langelier, M F et al., Nature Communications, 2018) using 25 μM inhibitors. The DNA binding affinity assay was also performed as described (10), using 5 μM inhibitors. The full-length WT PARP-1 protein used in these assays was purified as described previously (30).

### Expression and Purification of Proteins and Inhibitor Plate Assay

N-Terminal His-tagged human PARP-1 and SRPK2 was expressed and purified as previously described (Carter-O'Connell et al., 2014). PARP-1 was purified to greater than 90% and SRPK2 to 70% or greater by an in-gel standard curve of Bovine Serum Albumin (Bio-Rad). IC₅₀ SRPK2 Plate Assays were performed as previously described (21). Log(inhibitor) versus response curves were fit using non-linear regression with variable slope. Mean IC₅₀ values for each compound were derived from a minimum of three separate replicates.

### Cell Lines

The human embryonic kidney cell line (HEK 293T) and HeLa cell line was purchased from the American Type Culture Collection. Cells were maintained in DMEM (10% FBS, 1% Glutamine) at 37° C. with 5% CO₂ and passaged every 2-3 days. HEK 293T and Hela cells were periodically authenticated and tested for mycoplasma contamination. HEK 293 Control and PARP1 KO cells lines were gifted from Michael Garabedian at NYU Langone. The source, etiology, and culture conditions for ES-8 cells were previously reported (25)

### γH2AX Immunofluorescence

HEK 293T or HEK 293 PARP-1 KO cells were seeded onto poly-d-lysine treated glass coverslips and treated with indicated concentrations of inhibitors for 18 hours. Cells were then washed with 1×PBS and fixed using 4% Sucrose/4% Paraformaldehyde in PBS for 20 minutes. After three washes in 1×PBS, cells were permeabilized in 0.3% Triton X-100 in PBS for 5 minutes followed by an additional three washes in 1×PBS. Coverslips were blocked in 3% BSA/0.1% Triton X-100 in PBS for 1 hour and then placed in primary anti-rabbit phosphoH2Ax antibody (1:200, Cell Signaling) overnight at 4° C. The next morning, cells were washed three times in 1×PBS and treated with blocking solution, 3% BSA/0.1% Triton X-100 in PBS, for 30 minutes before being placed in secondary anti-rabbit Alexa Fluor 647 (1:1000 Jackson Immuno) for 1 hour at RT. After three subsequent 1×PBS washes, coverslips were mounted using ProLong Gold antifade reagent with DAPI (Invitrogen). All immunofluorescence images were taken on a Zeiss ApoTome2 on Axiolmager and analyzed using Zeiss software. Mean intensity of the Alexa Fluor 647 signal was measured after imposing DAPI masks over the nucleus of each cell. All cells within a 20× image, was analyzed (˜3000 cells/condition). Statistical significance was assigned using one-way ANOVA with multiple comparisons.

### Neutral Comet Assay

HEK 293T cells were treated overnight with 1 μM AZ0108 or 1 μM Olaparib, or treated for 40 minutes with 100 μg/ml bleomycin. Cells were washed with 1×PBS, trypsinized, resuspended in a 1000,000 cells/ml 1×PBS solution, and mixed with molten agarose 1:2. The agarose mixture was placed onto CometSlides and the comet assay was completed following the manufacturer instructions (Trevigen, 4250-050-K). DNA was stained using SYBR Green and slides were imaged on a Zeiss ApoTome2 on Axiolmager using a 10× objective (>100 cells/condition). Olive Moment was analyzed using CometScore software and statistical significance was assigned using one-way ANOVA with multiple comparisons.

### Western Blot Analysis of Phosphorylation of Chk1

HEK 293T cells were treated with the indicated concentrations of inhibitors for 18 hours at 37° C. Cells were washed with PBS, and lysed in RIPA buffer (50 mM Tris-HCl [pH 8.0], 150 mM NaCl, 0.1% SDS, 1% Triton X-100, 1× cOmplete™ protease inhibitors (Roche), Phosphatase inhibitors, and 1 μM Veliparib). Lysates were centrifuged at 10,000× rpm for 10 min at 4° C. and supernatants were transferred to a new tube and quantified via Bradford Assay (BioRad). Following quantification, 4× Laemmli sample buffer with 5% β-mercaptoethanol was added to 25-50 μg of protein. Samples were resolved by SDS-PAGE and transferred onto nitrocellulose membranes (BioRad Turbo Transfer System). Membrane blots were blocked with 5% milk-PBST for 1 h at RT, followed by incubation with a rabbit pChk1 antibody (Cell Signaling) or a mouse b-actin antibody (Santa Cruz Biotechnology) for 2 h at RT, followed by incubation with HRP-conjugated secondary antibodies. ECL HRP substrate (SuperSignal™ West Pico, ThermoFisher) was added to detect protein targets by chemiluminescence. Blots were imaged for chemiluminescent signal on a ChemiDoc MP system (BioRad). Statistical significance was assigned using one-way ANOVA with multiple comparisons.

### PARP6 Transcript Sequencing and Protein Identification

To make RNA samples, HEK 293T and HeLa cell pellets were resuspended with Trizol reagent (Invitrogen) and incubated on ice for 10 minutes. Samples were frozen at −80° C. until subsequent processing. When ready, samples were thawed on ice and then incubated at room temperature for 5 minutes. 1-Bromo-3-Chloropropane (Fisher Scientific) was added to each sample and incubated at room temperature for 2-3 minutes after vigorous shaking. Samples were centrifuged at 12000 g for 10 minutes at 4° C. The upper phase of the spun samples were transferred to a clean Eppendorf tube. Next, glycogen (RNA grade, Thermo Scientific) and 100% isopropanol was added at and the samples were incubated at room temperature for 10 minutes and then at −20° C. for 1 hour. Samples were then centrifuged at 12000 g for 10 minutes at 4° C. The supernatant was discarded and the pellet was washed with 75% ethanol. Samples were centrifuged at 7500 g for 5 minutes at 4° C. The ethanol wash was discarded and pellets were air dried for 15 minutes. The RNA pellets were resuspended in H₂O and incubated at 55° C. for 10 minutes. cDNA was created from the respective RNA by incubating 1000 ng of RNA with Oligo(dT) 18 Primer (Thermo Scientific) and Random Hexamers (50 μM, Applied Biosystems, Invitrogen) for 5 minutes at 65° C. Samples were then incubated with ProtoScript II reagents (First Strand cDNA Synthesis Kit, NEB) at 25° C. for 5 minutes, 42° C. for 1 hour, and 80° C. for 5 minutes. PARP6 transcripts were identified using primers designed to amplify full-length human PARP6: F 5′-GACTCAGCTAGCGGGCCAGTTCTGGA-3′ (SEQ ID NO: 1), R: 5′-TGAGTCGAATTCTCTGAGTTCCGATC-3′ (SEQ ID NO: 2). PCR fragments were processed via Phusion High-Fidelity DNA Polymerase (NEB) and deoxynucleotide solution (10 mM, NEB). PCR products were run out on an agarose gel, prepped, and sent for Sanger Sequencing.

For western blot analysis, cortical tissue from WT and PARP6 KO mice, and HEK 293T cells transfected with GFP-PARP6 or mock plasmid, were lysed in RIPA buffer (50 mM Tris-HCl [pH 8.0], 150 mM NaCl, 0.1% SDS, 1% Triton X-100, 1× complete protease inhibitors (Roche), Phosphatase inhibitors). Lysates were centrifuged at 10,000× rpm for 10 min at 4° C. and supernatants were transferred to a new tube and quantified via Bradford Assay (BioRad). Following quantification, 4× Laemmli sample buffer with 5%3-mercaptoethanol was added to 25-50 mg of protein. Samples were resolved by SDS-PAGE and transferred onto nitrocellulose membranes (BioRad Turbo Transfer System). Membrane blots were blocked with 5% milk-PBST for 1 hour (h) at RT, followed by incubation with a PARP6 antibody (gift from Dr. Paul Chang) or a mouse b-actin antibody (Santa Cruz Biotechnology) for 2 h at RT, followed by incubation with HRP-conjugated secondary antibodies. ECL HRP substrate (SuperSignal West Pico, ThermoFisher) was added to detect protein targets by chemiluminescence. Blots were imaged for chemiluminescent signal on a ChemiDoc MP system (BioRad).

### Cell Proliferation Assay

HEK 293T or HEK 293 PARP-1 KO cells were seeded in a 96 well plate at 1000-2000 cells per well. Following an overnight incubation, MTT reagent, 3-(4,5-dimethylthiazol-2-yl)-2,5-diphenyltetrazolium bromide, or a dosage curve of indicated inhibitor was added to each well in quadruplicate. Following a 3-hour incubation at 37° C., wells containing MTT reagent, were aspirated and lysed with 100 μl DMSO. These wells acting as a no growth control were collected, saved @ −20° C., and replaced with 100 μl water. The plate was placed back into the incubator for a total of 72 hours of inhibitor treatment prior to MTT reagent being added for a final time. Following a 3 hour incubation with the reagent, wells were aspirated, and 100 μl DMSO were added to each well, while the no growth control sample was added back to the plate. Absorbance was read on a SpectraMaxi3 plate reader (Molecular Devices). Percent growth was calculated for analysis. Statistical significance was assigned using individual t-tests between conditions for each inhibitor dosage.

### Cell Apoptosis Assay

HEK 293 or HEK 293 PARP-1 KO cells were seeded in a 96 well plate at 1000-2000 cells per well, a media only “blank” was also included. Following an overnight incubation, cells were treated with 1 μM of indicated inhibitors. “Blank” wells were treated with DMSO. Following a 72 hour inhibitor treatment, the Caspase-Glo 3/7 Assay was completed following the manufacturer instructions (Promega, #G8091). Luminescence was read on a SpectraMax i3 plate reader (Molecular Devices). Statistical significance was assigned using one-way ANOVA with multiple comparisons.

### Analysis of Cell Cycle

HEK 293T cells were treated with DMSO, 1 μM AZ0108, 1 μM Olaparib, or 2 mM Thymidine for 18 hours at 50% confluency. After the indicated treatments, cells were washed twice with 1× PBS, trypsinized off of the plate, and washed an additional 2 times with 1×PBS. Cells were then permeabilized using 70% cold ethanol while vortexing before incubating for 30 min at 4° C. Cells were then washed three times with 1×PBS and counted using a hemocytometer to create a final pellet of 1×10⁶ cells in for each inhibitor condition. 500 μl of DAPI solution (1 μg/ml DAPI, 0.1% Triton X-100 in PBS) was added to each cell pellet and incubated on ice for 30 min before being analyzed by flow cytometry using a BD Canto II. All data was analyzed on FlowJo software, gating for 20,000 singlet events. Manual cell cycle gates were set onto the DMSO control data using Watson modeling as reference. These gates were applied to all treatment conditions to obtain quantitative analysis.

### DNA Synthesis Assay

HEK 293T cells were seeded onto poly-d-lysine treated glass coverslips. Cells were then treated with DMSO, 1 μM AZ0108, 1 μM Olaparib, or 2 mM Thymidine for 18 hours. After incubation, cells were treated with 10 μM EdU (5-ethynyl-2′-deoxyuridine) for 1.5 hours and subsequently fixed with 4% Sucrose/4% Paraformaldehyde in PBS for 15 minutes at RT. Cells were washed 2× with 3% BSA in PBS and permeabilized with 0.3% Triton X-100 in PBS for 20 minutes at RT. After two additional 3% BSA in PBS washes, coverslips were incubated with 500 μl Click-iT reaction cocktail [1× Click-iT reaction buffer, 2 mM CuSO₄, 5 μM TAMRA Azide, Reaction buffer additive] for 30 minutes at room temperature (RT) protected from the light. Cells were washed once more with 3% BSA in PBS and twice more with 1×PBS before being mounted using ProLong Gold antifade reagent with DAPI (Invitrogen). All immunofluorescence images were taken on a Zeiss ApoTome2 on Axiolmager and analyzed using Zeiss software. Mean intensity of the TAMRA signal was measured after imposing DAPI masks over the nucleus of each cell. All cells within a 20× image, was analyzed (˜3000 cells/condition). Statistical significance was assigned using one-way ANOVA with multiple comparisons.

### Computational Inhibitor Docking and Dynamics

The human PARP-1 crystal structure (4DQY) bound to DNA was not allowing the inhibitor (AZ0180) to dock properly as the D-loop region was occluding the catalytic domain pocket. To facilitate the docking, we generated a PARP-1 model of ART and HD domain based on the human PARP2 crystal structure (4TVJ) bound to Olaparib. The highest sequence homology (similarity>68%) allowed to generate quality PARP-1 structure to Induced-Fit-Docking (IFD) (Schrödinger, LLC, New York, NY, 2013). After adding the hydrogen atoms, the protein structure was subjected to short minimization using OPLS_2005 force-filed, the IFD was performed in the extended mode generating more than 80 poses per ligand. The IFD protocol initially uses GLIDE protocol to generate ligand conformations in the protein GRID that cover catalytic domain and 20 Å surrounding, following that each pose is refined by allowing ligand and the 5 Å residues coordinating the ligand interaction move. Finally, energetically refined complex poses were reported based on the IFD energy and docking score.

The system preparation includes assigning histidine protonation using REDUCE program. The simulation scripts were generated using CHARMM-GUI. The scripts add hydrogen, solvate, neutralize, and add periodic boundary conditions. The apo and complex structures were solvated using TIP3P water molecules in a cubic box and chloride ions were added to neutralize the system. The CHARMM22/CMAP all-atom additive force field used to simulate the system, AZ0108 parameters obtained from CHARMM General Force Field. The molecular dynamics (MD) simulation performed in NAMD 2.10. The non-bonded interactions (Lennard-Jones) truncated using the force switching function between 10.0 and 12.0 Å, non-bonded pair list generation restricted to 16.0 Å and the list was periodically updated. The particle mesh Ewald (PME) method was used to treat long-range electrostatic interactions. The SHAKE method was applied to constrain the covalent bonds involving hydrogen atoms. A 2 fs integration time-step was used during the simulation. The simulation system subjected to 10000 steps minimization using the conjugate gradient method, 500 ps equilibration was carried in the NVT ensemble at 303.15 K. Following that production run was performed in the NPT ensemble, Langevin Piston method was used to maintain temperature (303.15 K) and pressure (1 atm). The initial velocities were assigned randomly according to Maxwell distribution. Both apo and complex systems were simulated to 200 ns. The simulation trajectories were analyzed in CHARMM MD package, VMD, and in-house scripts.

### Cloning

PARP-1 D770A and R878A were cloned using gBlock gene fragments containing the PARP-1 gene as template (IDT) and the following primers (IDT) for subsequent cloning: (D770A) F 5′-AGCTAGGCTGAGCAAAAGGCAGATC-3′ (SEQ ID NO: 3), R 5′-(SEQ CTAGCTGATATCGACTTCCAAGTCAT-3′ AGCTAGGATATCTCGTGAAGGCGAAT-3′ (SEQ ID NO: 5), R 5′-ID NO: 4); (R878A) F 5′-CTAGCTACCGGTGGATCCCGGGA-3′ (SEQ ID NO: 6). The amplified D770A and R878A fragments were gel purified and digested using Eco-RV/Blpl or Eco-RV/Agel restriction enzymes respectively. Mutant fragments were ligated into the mCherry-PARP-1 backbone, a generous gift from Gyula Timinszky at the Hungarian Academy of Sciences.

### PARP-1 Auto-PARylation Activity Assay in Cells

HEK 293T cells were pre-treated with the indicated concentrations of inhibitor for 30 min at 37° C., followed by incubation with PDD 00017273 (10 μM) for an additional 30 min. Cells were washed with PBS, and lysed in RIPA buffer (50 mM Tris-HCl [pH 8.0], 150 mM NaCl, 0.1% SDS, 1% Triton X-100, 1× cOmplete™ protease inhibitors (Roche), and 1 μM Veliparib). Lysates were centrifuged at 10,000× rpm for 10 min at 4° C. and supernatants were transferred to a new tube and quantified via Bradford Assay (BioRad). Following quantification, 4× Laemmli sample buffer with 5% β-mercaptoethanol was added to 25-50 μg of protein. Samples were resolved by SDS-PAGE and transferred onto nitrocellulose membranes (BioRad Turbo Transfer System).

Membrane blots were blocked with 5% milk-PBST for 1 h at RT, followed by incubation with a rabbit pan-ADP-ribose binding reagent (Cell Signaling), a rabbit PARP-1 antibody (Bethyl Laboratories), or a mouse b-actin antibody (Santa Cruz Biotechnology) for 2 h at RT, followed by incubation with HRP-conjugated secondary antibodies. ECL HRP substrate (SuperSignal™ West Pico, ThermoFisher) was added to detect protein targets by chemiluminescence. Blots were imaged for chemiluminescent signal on a ChemiDoc MP system (BioRad). Statistical significance was established through multiple unpaired t-tests with nonlinear regression fit analysis.

For off-rate analysis, after 30 minute pre-treatment with indicated inhibitors, cells were washed once before incubating for 0, 3, 6, or 12 hours in fresh media. Following release times, cells were incubated with PDD 00017273 (10 μM) for 30 minutes before proceeding using the same western blot protocol detailed above. Statistical significance was established through multiple unpaired t-tests with nonlinear regression fit analysis.

To analyze catalytic activity of PARP-1 D770A and R878A mutants, HEK 293T cells were transfected with 3 μg of mCherry-PARP-1 WT, mCherry-PARP-1 D770A, or mCherry-PARP-1 R₈₇₈A using CalPhos transfection reagent. Cells were lysed in a cytosolic lysis buffer (50 mM HEPES pH 7.4, 150 mM NaCl, 1 mM MgCl₂, 1% Triton X-100, 1× cOmplete™ protease inhibitors (Roche), 100 μM TCEP (tris(2-carboxyethyl)phosphine)). Lysates were centrifuged at 10,000× rpm for 10 min at 4° C. and supernatants were transferred to a new tube and quantified via Bradford Assay (BioRad). Following quantification, 1 mg protein was bound onto RFP-Trap beads (Chromotek) at 4° C. for 1 hour while rotating. Beads were then washed 3 times with lysis buffer and once with high salt wash (lysis buffer with 500 mM NaCl). Beads were then incubated in PARP reaction buffer (50 mM Tris-HCl, 50 mM NaCl, 0.1% Triton X-100, 1× cOmplete™ protease inhibitors (Roche), 100 μM TCEP) in the presence of 100 μM 6-alkyne NAD⁺ with or without 3 μM AZ0108 for 1.5 hours at 25° C. with 650 rpm shaking. Beads were subsequently washed twice with PARP reaction buffer, once with 1×PBS, and then incubated with click reaction mix (1 mM CuSO₄, 100 μM biotin-azide, 100 μM TBTA (Tris((1-benzyl-4-triazolyl)methyl)amine), 1 mM TCEP in PBS) for 1.5 hours at 25° C. with 650 rpm shaking. After final incubation, 1.5× Laemmli sample buffer with 5% β-mercaptoethanol was added to each bead sample and boiled for 10 minutes at 95° C. Samples were resolved by SDS-PAGE and transferred onto nitrocellulose membranes (BioRad Turbo Transfer System). Membrane blots were blocked with 5% milk-PBST for 1 h at RT, followed by incubation with either a streptavidin-HRP antibody (Jackson ImmunoResearch) for 45 minutes at RT or a rabbit PARP-1 antibody (Bethyl Laboratories) and mouse b-actin antibody (Santa Cruz Biotechnology) for 2 hours at RT followed by incubation with HRP-conjugated secondary antibodies. ECL HRP substrate (SuperSignal™ West Pico, ThermoFisher) was added to detect protein targets by chemiluminescence. Blots were imaged for chemiluminescent signal on a ChemiDoc MP system (BioRad). Statistical significance was assigned using unpaired t-test.

### Photoaffinity Labelling

In vitro photocrosslinking. Recombinant PARP-1 the was immunoprecipitated with PARP-1-Trap agarose beads (Chromotek) as follows: Three hundred nM of protein in 100 μL hB (100 mM NaCl, 50 mM HEPES, 4 mM MgCl₂, pH 7.5) was incubated with 5 μL beads and tumbled end-over-end for 30 min. Unbound protein was removed by centrifuging the suspension at 2500× g for 2 min, the supernatant was carefully removed and the pellet was resuspended in 100 μLhB. Samples were then treated with 1% DMSO, 10 M AZ0108 or 10 μMM PIP6 tumbled end-over-end for 30 min. For washout condition the following was repeated 3 times: beads were suspended in 0.5 mL hB, incubated for 5 min, spun 2500× g and supernatant was carefully removed. After the final wash, beads were resuspended in 100 μL hB. All samples were then treated with 0.1 μM 933 for 8 min and irradiated for 90 s in the RPR-100 Photochemical Reactor (Rayonet) equipped with 350 nm lamps at 4° C. The optimal compound concentrations and UV time was determined beforehand (data not shown). All steps were performed RT, except if otherwise noted. Twenty-five μl of each sample were mixed with fatty acid free BSA and SDS to give a final Click reaction concentration of 1 mg/mL and 1%, respectively.

Photocrosslinking in intact cells. HEK293 CRL and PARP-1 KO cells were grown in DMEM for 2 days in a 60 mm dish for 2 days to reach 80-90% confluency. On the day of the experiment the media was replaced and i) washout condition: cells were treated with 0.1% DMSO, 10 PM AZ0108 or 10 μM PIP6 for 30 min 37° C., then the media was replaced again and cells were incubated at 37° C. for 2 h before treatment with 0.1 μM ad-olaparib for additional 1 h; ii) no washout condition: cells were treated with 0.1 μM ad-olaparib for 30 min and then 0.1% DMSO, 10 PM AZ0108 or 10 UM PIP6 were added for additional 30 min co-treatment at 37° C. Cells were then washed once with PBS, covered with PBS and irradiated for 5 min in the RPR-100 Photochemical Reactor (Rayonet) equipped with 350 nm lamps at 4° C. Cells were lysed on ice in 1% SDS, 1% Triton X-100 and EDTA-free protease inhibitor cocktail (Roche) in PBS for 10 min and then probe sonicated to shear the nuclear DNA. Protein concentration was determined with Bio-Rad Protein assay. The lysate was diluted in PBS to a final Click reaction concentration of 2 mg/mL.

Click Chemistry. Reaction was started by mixing 30 μL of each sample with 15 μL of PBS based 3× Click reaction mixture containing (final concentrations): 100 μM Tris[(1-benzyl-1H-1,2,3triazol-4-yl)methyl]amine (TBTA, Click Chemistry Tools), 1 mM CuSO₄, 40 μM tetramethylrhodamine (TAMRA)-azide (Click Chemistry Tools), 1 mM Tris(2-carboxyethyl)phosphine hydrochloride (TCEP, Thermo Fisher) and 15% tert-Butyl alcohol, and let to procced for 1 h at RT while gently shaking. Reactions were stopped by adding 4× Laemmli sample buffer and boiling at 95° C. for 5 min.

In gel fluorescence, western blotting and quantification. Samples were loaded to 8 or 10% SDS-polyacrylamide gels and run until the dye front left the gel. First, in-gel fluorescence was detected with ChemiDoc XRS+ (Biorad) using Rhodamine filter and then the proteins were transferred on nitrocellulose membrane, the membrane was blocked and probed for PARP-1 (1:3000) and alpha-tubulin (1:1000). Both antibodies were from cell signaling. Band intensities we quantified with ImageJ.

### ES-8 Viability Screen

Cells were plated in 96-well assay plates (Corning, 3917) at a density of 1000 cells/well, allowed to equilibrate for 24 h, then exposed to drug for 72 h. Cell viability was measured using the CellTiter-Glo (Promega, G7573) assay and read using an Envision (PerkinElmer) plate reader. Compound source plates (96-well) were prepared by solubilizing each drug in DMSO to a maximum concentration of 1-50 mM, then arraying samples in 10-point dose-response with 3-fold dilution. Positive and negative controls (staurosporine at 10 mM and DMSO, respectively) were added to the source plates. Drug was transferred from the source plate to the assay plate using a Biomek FX (Beckman Coulter) liquid handler equipped with a pin tool which transferred ˜100 nl into a final volume of 100 μl, resulting in ˜1000-fold dilution. At least two biological replicates (assay performed on different day with same drug plate and independent cell preparation) were obtained for each drug. To analyze the assay, the relative light unit signal in each well was first log 2 transformed, then normalized by subtracting the mean of the negative controls wells on each plate. The Hill parameters were estimated using the drm function in the R drc package (Ritz et al, PLOS ONE, 2015; R Core Team (2019). R: A language and environment for statistical computing. R Foundation for Statistical Computing, Vienna, Austria. URL https://www.R-project.org/) with the following constraints: 0>hill slope>10, y₀=0; −10<yₘₐₓ<0; 10⁻¹¹<EC₅₀<10⁻³.

### Statistical Analysis

Beyond individualized analysis within each assay methodology, all data was processed using GraphPad Prism version 9.0. Data was analyzed using one-way ANOVA, unless stated otherwise, and considered statistically significant if p<0.05. All data was presented as a mean +/−standard error of the mean (SEM).

### Chemical Synthesis

General chemistry methods. ¹H NMR spectra were recorded on a Bruker DPX spectrometer at 400 MHz. Chemical shifts are reported as parts per million (ppm) downfield from an internal tetramethylsilane standard or solvent references. For air- and water-sensitive reactions, glassware was flame- or oven-dried prior to use and reactions were performed under argon. Dimethylformamide was dried using the solvent purification system manufactured by Glass Contour, Inc. (Laguna Beach, CA). All other solvents were of ACS chemical grade (Fisher Scientific) and used without further purification unless otherwise indicated. Commercially available starting reagents were used without further purification. Analytical thin-layer chromatography was performed with silica gel 60 F254 glass plates (SiliCycle). Flash column chromatography was conducted self-packed columns containing 200-400 mesh silica gel (SiliCycle) on a Combiflash Companion purification system (Teledyne ISCO). High performance liquid chromatography (HPLC) was performed on a Varian Prostar 210 (Agilent) using Polaris 5 C18-A columns (Analytical: 150×4.6 mm, 3 μm; Preparative: 150×21.2 mm, 5 μm) (Agilent). Low-resolution mass spectra (LR-MS) were acquired on an Advion Mass-express.

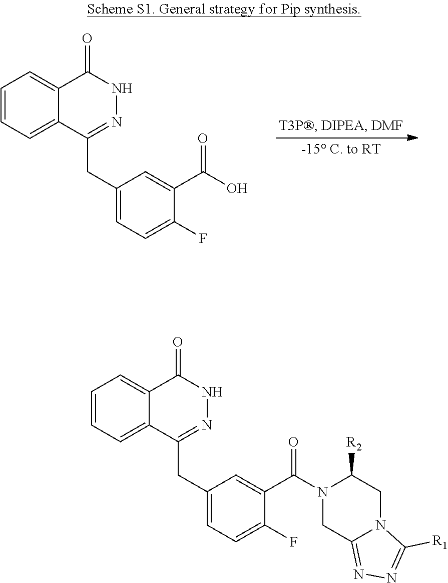

General strategy for amide coupling reaction (Scheme S1). 2-fluoro-5-((4-oxo-3,4-dihydrophthalazin-1-yl)methyl)benzoic acid (Ark Pharm) (0.34-0.65 mmol) and the indicated triazolo[4,3-a]pyrazines (1.0 eq.) were added to a flame dried flask and dissolved in anhydrous DMF (3 mL). DIPEA (0.3 mL, 1.7 mmol) was added under argon and the reaction cooled to −15° C. and stirred for 10 min. 1-propanephospohinic acid cyclic anhydride (T3P®) (50% in DMF, Acros Organics) (0.4 mL, 0.68 mmol) was added dropwise to the reaction mixture under argon and the reaction stirred for 10 min at −15° C., then slowly warmed to RT and stirred for 18 h. The reaction mixture was then poured over water (100 mL) and extracted with DCM (3×40 mL). The combined organic layers were washed with water (1×100 mL) and brine (1×100 mL) and concentrated in vacuo followed by purification via ISCO Combiflash chromatography (silica, 4 g, 0-10% MeOH in DCM).

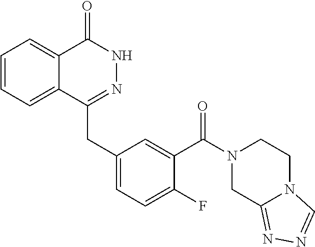 [SMILES: O=C(c1cc(Cc2n[nH]c(=O)c3ccccc23)ccc1F)N1CCn2cnnc2C1]

4-(4-Fluoro-3-(5,6,7,8-tetrahydro-[1,2,4]triazolo[4,3-a]pyrazine-7-carbonyl)benzyl)phthalazin-1(2H)-one, (Pip1). From 5,6,7,8-tetrahydro-[1,2,4]triazolo[4,3-a]pyrazine hydrochloride (54 mg, 0.34 mmol). Yield: 40 mg (29%) white solids. ¹H NMR (400 MHZ, DMSO-d6) δ 12.58 (s, 1H), 8.51 (s, 1H), 8.27 (d, J=7.8 Hz, 1H), 7.97 (d, J=8.4 Hz, 1H), 7.94-7.87 (m, 1H), 7.87-7.80 (m, 1H), 7.53-7.41 (m, 2H), 7.33-7.24 (m, 1H), 4.94 (s, 1H), 4.60 (s, 1H), 4.14 (d, J=5.4 Hz, 1H), 4.08 (d, J=4.1 Hz, 1H), 4.00 (t, J=5.5 Hz, 1H), 3.63 (s, 1H). LR-MS m/z [M+H]+ for C₂₁H₁₇FN₆O₂ 405.14, calculated 405.2.

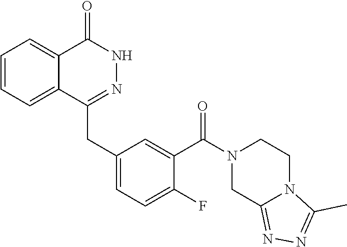 [SMILES: Cc1nnc2n1CCN(C(=O)c1cc(Cc3n[nH]c(=O)c4ccccc34)ccc1F)C2]

4-(4-Fluoro-3-(3-methyl-5,6,7,8-tetrahydro-[1,2,4]triazolo[4,3-a]pyrazine-7-carbonyl)benzyl) phthalazin-1(2H)-one (Pip2). From 3-methyl-5,6,7,8-tetrahydro-[1,2,4]triazolo[4,3-a]pyrazine (46 mg, 0.34 mmol). Yield: 38 mg (27%) white solids. ¹H NMR (400 MHZ, DMSO-d6) δ 12.58 (s, 1H), 8.27 (d, J=7.6 Hz, 1H), 7.99-7.80 (m, 3H), 7.49 (s, 1H), 7.43 (d, J=6.7 Hz, 1H), 7.29 (t, J=8.9 Hz, 1H), 4.87 (s, 1H), 4.54 (s, 1H), 4.34 (s, 2H), 4.08 (s, 1H), 3.98 (s, 1H), 3.70 (s, 1H), 3.59 (s, 1H), 2.29 (d, J=15.9 Hz, 3H). LR-MS m/z [M−H]− calculated for C₂H₁₉FN₆O₂ 417.16, observed 416.7.

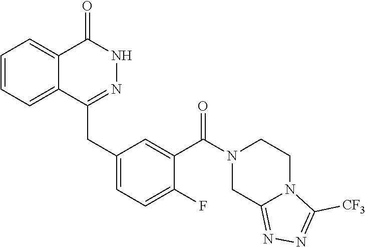 [SMILES: O=C(c1cc(Cc2n[nH]c(=O)c3ccccc23)ccc1F)N1CCn2c(nnc2C(F)(F)F)C1]

4-(4-Fluoro-3-(3-(trifluoromethyl)-5,6,7,8-tetrahydro-[1,2,4]triazolo[4,3-a]pyrazine-7-carbonyl) benzyl) phthalazin-1(2H)-one (Pip3). From 3-(trifluoromethyl)-5,6,7,8-tetrahydro-[1,2,4]triazolo[4,3-a]pyrazine (65 mg, 0.34 mmol). Yield: 50 mg (31%) white solids. ¹H NMR (400 MHz, DMSO-d6) δ12.58 (s, 1H), 8.26 (dd, J=7.3, 1.6 Hz, 1H), 7.97-7.79 (m, 3H), 7.52 (d, J=8.4 Hz, 1H), 7.44 (d, J=6.3 Hz, 1H), 7.30 (t, J=8.8 Hz, 1H), 5.02 (s, 1H), 4.70 (s, 1H), 4.34 (s, 2H), 4.23 (s, 1H), 4.14 (s, 1H), 3.97 (s, 1H), 3.67 (s, 1H). LR-MS m/z [M−H]− calculated for C₂₂H₁₆F₄N₆O₂ 471.4, observed 471.0.

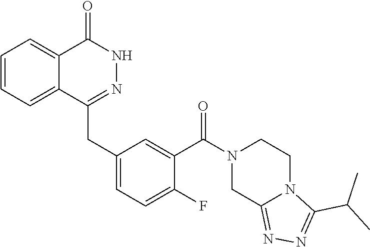 [SMILES: CC(C)c1nnc2n1CCN(C(=O)c1cc(Cc3n[nH]c(=O)c4ccccc34)ccc1F)C2]

4-(4-Fluoro-3-(3-isopropyl-5,6,7,8-tetrahydro-[1,2,4]triazolo[4,3-a]pyrazine-7-carbonyl)benzyl) phthalazin-1(2H)-one (Pip4). From 3-isopropyl-5,6,7,8-tetrahydro-[1,2,4]triazolo[4,3-a]pyrazine (56 mg, 0.34 mmol). Yield: 60 mg (40%) white solids. ¹H NMR (400 MHZ, DMSO-d6) δ 12.58 (s, 1H), 8.26 (d, J=7.6 Hz, 1H), 7.98-7.79 (m, 3H), 7.52-7.42 (m, 2H), 7.30 (t, J=8.9 Hz, 1H), 4.88 (s, 1H), 4.55 (d, J=3.4 Hz, 1H), 4.34 (s, 2H), 4.09 (q, J=5.2 Hz, 1H), 3.75 (s, 1H), 3.58 (s, 1H), 3.17 (dd, J=5.3, 0.9 Hz, 2H), 2.14-2.06 (m, 1H), 1.24 (d, J=6.8 Hz, 6H). LR-MS m/z [M−H]− calculated for C₂₄H₂₃FN₆O₂ 445.2, observed 444.9.

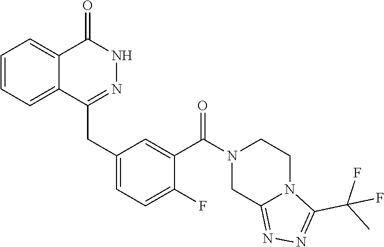 [SMILES: CC(F)(F)c1nnc2n1CCN(C(=O)c1cc(Cc3n[nH]c(=O)c4ccccc34)ccc1F)C2]

4-(4-Fluoro-3-(3-(1,1-difluoroethyl)-5,6,7,8-tetrahydro-[1,2,4]triazolo[4,3-a]pyrazine-7-carbonyl) benzyl)phthalazin-1(2H)-one (Pip5). From 3-(1,1-difluoroethyl)-5,6,7,8-tetrahydro-[1,2,4]triazolo[4,3-a]pyrazine (synthesized according to (12) with slight modifications, (70 mg, 0.37 mmol). Yield: 60 mg (46%). ¹H NMR (400 MHZ, CDCl₃) δ 11.08 (d, J=50.8 Hz, 1H), 8.46 (d, J=6.7 Hz, 1H), 7.84-7.67 (m, 3H), 7.44-7.36 (m, 2H), 7.09 (t, J=9.1 Hz, 1H), 5.15 (s, 1H), 4.81 (s, 1H), 4.44-4.08 (m, 5H), 3.75 (s, 1H), 2.23 (t, J=19.2 Hz, 3H). LR-MS (ESI) m/z [M]+ calculated for C₂₃H₁₉F₃N₆O₂ 468.43, observed 469.0.

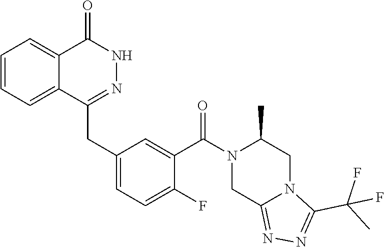 [SMILES: C[C@H]1Cn2c(nnc2C(C)(F)F)CN1C(=O)c1cc(Cc2n[nH]c(=O)c3ccccc23)ccc1F]

(S)-4-(3-(3-(1,1-difluoroethyl)-6-methyl-5,6,7,8-tetrahydro-[1,2,4]triazolo[4,3-a]pyrazine-7-carbonyl)-4-fluorobenzyl)phthalazin-1(2H)-one (Pip6). From (S)-3-(1,1-difluoroethyl)-6-methyl-5,6,7,8-tetrahydro-[1,2,4]triazolo[4,3-a]pyrazine (113 mg, 0.55 mmol) (12). Yield: 171.3 mg (65%). ¹H NMR (400 MHZ, CDCl₃) δ 11.35 (d, J=72.2 Hz, 1H), 8.47 (d, J=6.5 Hz, 1H), 7.94-7.59 (m, 3H), 7.39 (dt, J=10.9, 5.7 Hz, 2H), 7.10 (t, J=8.5 Hz, 1H), 5.93-5.34 (m, 1H), 5.00-4.42 (m, 2H), 4.29 (d, J=21.0 Hz, 4H), 2.23 (t, J=19.2 Hz, 3H), 1.46-1.06 (m, 3H). LR-MS (ESI) m/z [M]⁺ calculated for C₂₄H₂₁F₃N₆O₂ 482.46, observed 483.0.

### Figure Descriptions αF

FIGS. 1A-1D. AZ0108 is a type I inhibitor of PARP-1.

FIG. 1A) Theoretical representation of PARP-1-DNA binding allostery FIG. 1B) Chemical structure of PARP-1 inhibitors, Olaparib and AZ0108. Colors correspond to panel a. FIG. 1C) PARP-1-DNA release. PARP-1 (40 nM) was incubated with a dumbbell DNA probe carrying an internal FAM group and a central nick at 20 nM with or without compound (25 μM) for 30 minutes. An unlabeled competitor was added and the decrease in fluorescence polarization was measured across time. FIG. 1D) The K_{d} of PARP-1 for a DNA dumbbell probe containing a central nick (5 nM) was measured in a fluorescence polarization assay with and without inhibitor (5 μM). Error bars represent Standard Error of the Mean (SEM) (n=3). Statistical significance calculated via one-way ANOVA with Tukey's multiple comparisons test (p<0.05).

### FIGS. 2A-2E. AZ0108 Induces Dose-Responsive Pan-Nuclear γH₂AX Dependent on PARP-1 Presence, PARP-1-DNA Affinity, and ATM/Jnk Pathways

FIG. 2A) Representative images of HEK293T cells treated with indicated concentration of AZ0108. Cell are stained for DAPI and γH₂AX. Scale bar indicates 50 μm. FIG. 2B) Quantification of panel a. Mean intensity of γH₂AX signal within DAPI masks analyzed using Zen software (n>2000 cells over at least 2 replicates). Values are normalized to the average DMSO control within each replicate. Error bars represent SEM. Statistical significance calculated via one-way ANOVA with Tukey's multiple comparisons test (p<0.05). FIG. 2C) HEK293 control or HEK293 PARP-1 KO cells were treated with or without 3 μM AZ0108. Mean intensity of γH₂AX signal within DAPI masks analyzed using Zen software (n>1300 cells over 3 replicates). Values are normalized to the average DMSO control within each replicate. Error bars represent SEM. Statistical significance was calculated via one-way ANOVA with Tukey's multiple comparisons test (p<0.05). FIGS. 2D, 2E) HEK293T cells were treated with indicated inhibitors. Mean intensity of γH₂AX signal within DAPI masks analyzed using Zen software (n>2800 cells over 2 replicates). Values are normalized to the average DMSO control within each replicate. Error bars represent SEM. Statistical significance calculated via one-way ANOVA with Tukey's multiple comparisons test (p<0.05).

### FIGS. 3A-3F. AZ0108 Treatment Induces Cellular Senescence in HEK 293T Cells

FIGS. 3A, 3B, 3C) HEK 293T cells or HEK 293 Control and PARP-1 KO cells were seeded in 96-well plate (1000 or 2000 cells/well respectively) and treated with indicated dosage curve of AZ0108 or Olaparib for 72 hours. MTT reagent was added to each well and absorbance was read after 3 hours of incubation. % growth was calculated from the raw absorbance values and statistical significance was calculated via multiple t-tests (n=3, p<0.05). Error bars represent SEM. FIG. 3D) HEK 293 Control or PARP-1 KO cells were seeded in 96-well white opaque plate (2000 cells/well) and treated with indicated with 1 μM or Olaparib or AZ0108 for 72 hours. Caspase-Glo 3/7 reagent was added to each well and luminescence was read after 30 minutes. Normalized luminescence was calculated for each trial (Control n=2, KO n=4) and statistical significance was obtained via one-way ANOVA with Tukey's multiple comparisons test (p<0.05). Error bars represent SEM. FIG. 3E) HEK 293T cells at 50% confluence were treated with indicated concentrations of inhibitors for 24 hours. Cell cycle was analyzed by flow cytometry (20,000 singlet events) and cell cycle statistics were calculated in FlowJo software against DMSO Watson modeling (n=3). FIG. 3F) DNA synthesis was measured using EdU incorporation. HEK 293T cells were treated with indicated concentration of PARP-1 inhibitor for 18 hours before adding 10 μM EdU to each well for 1.5 hours incubation. EdU incorporation was labelled using TAMRA azide click-chemistry and imaged at 20×. Mean intensity of TAMRA signal within DAPI masks analyzed using Zen software (n>3900 cells over 2 replicates). Values are normalized to the average DMSO control within each replicate. Error bars represent SEM. Statistical significance calculated via one-way ANOVA with Tukey's multiple comparisons test (p<0.05).

### FIGS. 4A-4E. The AZ0108 Binding Mode and the Dynamics of PARP-1

FIG. 4A) View of AZ0108 binding at the catalytic domain of PARP-1, green color represents the ART and pink is a helical domain, AZ0108 is shown in orange ball and stick model, the residues around the ligand is in stick model and the dotted line indicates hydrogen bond. FIG. 4B) Helical domain (pink cartoon) movement observed during the simulation, the red arrows indicate length and direction of movement. FIG. 4C) The residue-residue distance observed between the Ca atoms R878-D770 and Y889-V758, the increased distance sampling corresponds to the helical domain movement. FIG. 4D) PARP-1 WT, D770A, or R878A (40 nM) was incubated with a dumbbell DNA probe carrying an internal FAM group and a central nick at 20 nM with or without compound (25 μM) for 30 minutes. An unlabeled competitor was added and the decrease in fluorescence polarization was measured across time. FIG. 4E) The K_{d} of PARP-1 WT, D770A, or R878A for a DNA dumbbell probe containing a central nick (5 nM) was measured in a fluorescence polarization assay with and without inhibitor (5 μM). Error bars represent Standard Error of the Mean (SEM) (n=3). Statistical significance calculated via one-way ANOVA with Tukey's multiple comparisons test (p<0.05).

FIGS. 5A-5F. Analogue development uncovers scaffolding responsible for PARP-1-DNA affinity FIG. 5A) Chemical structure of synthesized PARP-1 inhibitors. FIG. 5B) PARP-1 (40 nM) was incubated with a dumbbell DNA probe carrying an internal FAM group and a central nick at 20 nM with or without compound (25 μM) for 30 minutes. An unlabeled competitor was added and the decrease in fluorescence polarization was measured across time. FIG. 5C) HEK293T cells were treated with indicated inhibitors. Mean intensity of γH₂AX signal within DAPI masks analyzed using Zen software (n>2800 cells over 2 replicates). Values are normalized to the average DMSO control within each replicate. Error bars represent SEM. Statistical significance calculated via one-way ANOVA with Tukey's multiple comparisons test (p<0.05). FIG. 5D) HEK 293T cells were seeded in 96-well plate (1000 cells/well) and treated with indicated inhibitors for 72 hours. MTT reagent was added two each well and absorbance was read after 3 hours of incubation. % growth was calculated from the raw absorbance values and statistical significance was calculated via multiple t-tests (n>2, p<0.05). Error bars represent SEM. FIG. 5E) Auto-PARylation of PARP-1 across dosage curve of AZ0108 and Pip 6. HEK 293T cells were pre-treated with indicated concentration of inhibitor for 30 minutes before incubation with PARGi (10 μM) for another 30 minutes. Cells were subsequently lysed using RIPA buffer, run out on SDS-PAGE, and probed for ADPr (Cell Signaling) and PARP-1 (Bethyl Laboratories). FIG. 5F) Quantification of western blot images. ADPr values are normalized to PARP-1 within each replicate and plotted as percentage compared to DMSO. Error bars indicate SEM (n=3). Statistical significance of dose curves calculated via nonlinear regression analysis indicating IC₅₀ values of 4.249e-008 and 2.261e-009 M respectively.

FIGS. 6A-6E. Off-rate kinetics of Pip6 translates to anti-proliferation and cytotoxicity in HEK293 and ES-8 cells FIG. 6A) HEK 293T cells were treated with DMSO, 10 μM AZ0108, or 10 μM Pip6 for 30 minutes before incubating in fresh media washout for 3 hours. 0.1 μM ad-olaparib (chemical structure indicated in panel a) was added to the cells for 1 hour before undergoing a UV treatment for 5 minutes in cold PBS. Cells were lysed and sonicated in PBS (1% Triton X-100+1% SDS), clicked with TAMRA-azide and run out on SDS-PAGE. The gel was imaged for TAMRA and probed for PARP-1 (Bethyl Laboratories), and alpha-tubulin (X). FIGS. 6B, 6D) Quantification of western blot images. TAMRA or ADPr values are normalized to PARP-1 within each replicate and plotted as percentage compared to DMSO. Error bars indicate SEM (n=3-4). Statistical significance of off-rate calculated via multiple t-tests (p<0.05). FIG. 6C) 300 nM of AZ0108 or Pip 6 for 30 minutes before incubating in fresh media for 0, 3, 6, or 12 hours. Cells were subsequently treated with PARGi (10 μM) for 30 minutes, lysed using RIPA buffer, run out on SDS-PAGE, and probed for ADPr (Cell Signaling) and PARP-1 (Bethyl Laboratories). FIG. 6E) ES-8 cells were treated with indicated concentration of inhibitors for 72 hours before measuring cell viability using the CellTiter-Glo kit (Promega G7573). IC₅₀ values were calculated using R statistical software.

### REFERENCES

1. M.-F. Langelier, T. Eisemann, A. A. Riccio, J. M. Pascal, PARP family enzymes: regulation and catalysis of the poly(ADP-ribose) posttranslational modification. Curr Opin Struc Biol 53, 187-198 (2018).
2. D. Slade, PARP and PARG inhibitors in cancer treatment. Gene Dev 34, 360-394 (2020).
3. H. Farmer, et al., Targeting the DNA repair defect in BRCA mutant cells as a therapeutic strategy. Nature 434, 917-921 (2005).
4. H. E. Bryant, et al., Specific killing of BRCA2-deficient tumours with inhibitors of poly(ADP-ribose) polymerase. Nature 434, 913-917 (2005).
5. J. Murai, et al., Trapping of PARP1 and PARP2 by Clinical PARP Inhibitors. Cancer Res 72, 5588-5599 (2012).
6. C. J. Lord, A. Ashworth, PARP inhibitors: Synthetic lethality in the clinic. Science 355, 1152-1158 (2017).
7. M.-F. Langelier, J. M. Pascal, PARP-1 mechanism for coupling DNA damage detection to poly(ADP-ribose) synthesis. Curr Opin Struc Biol 23, 134-143 (2013).
8. M.-F. Langelier, J. L. Planck, S. Roy, J. M. Pascal, Structural Basis for DNA Damage-Dependent Poly(ADP-ribosyl)ation by Human PARP-1. Science 336, 728-732 (2012).
9. J. M. Dawicki-McKenna, et al., PARP-1 Activation Requires Local Unfolding of an Autoinhibitory Domain. Mol Cell 60, 755-768 (2015).
10. M.-F. Langelier, L. Zandarashvili, P. M. Aguiar, B. E. Black, J. M. Pascal, NAD+ analog reveals PARP-1 substrate-blocking mechanism and allosteric communication from catalytic center to DNA-binding domains. Nature communications 9, 844 (2018).
11. L. Zandarashvili, et al., Structural basis for allosteric PARP-1 retention on DNA breaks. Science 368, eaax6367 (2020).
12. J. W. Johannes, et al., Discovery of AZ0108, an orally bioavailable phthalazinone PARP inhibitor that blocks centrosome clustering. Bioorg Med Chem Lett 25, 5743-5747 (2015).
13. E. Moeglin, et al., Uniform Widespread Nuclear Phosphorylation of Histone H2AX Is an Indicator of Lethal DNA Replication Stress. Cancers 11, 355 (2019).
14. N. P. Singh, M. T. McCoy, R. R. Tice, E. L. Schneider, A simple technique for quantitation of low levels of DNA damage in individual cells. Exp Cell Res 175, 184-191 (1988).
15. A. N. Blackford, S. P. Jackson, ATM, ATR, and DNA-PK: The Trinity at the Heart of the DNA Damage Response. Mol Cell 66, 801-817 (2017).
16. C. Lu, et al., Cell apoptosis: requirement of H2AX in DNA ladder formation, but not for the activation of caspase-3. Molecular cell 23, 121-132 (2006).
17. T. Ubhi, G. W. Brown, Exploiting DNA Replication Stress for Cancer Treatment. Cancer Res 79, 1730-1739 (2019).
18. Z. Wang, et al., Pharmacological inhibition of PARP6 triggers multipolar spindle formation and elicits therapeutic effects in breast cancer. Cancer Res 78, canres. 1362.2018 (2018).
19. A. Salic, T. J. Mitchison, A chemical method for fast and sensitive detection of DNA synthesis in vivo. Proc National Acad Sci 105, 2415-2420 (2008).
20. C. Gu, et al., Modulating the strength of hydrogen bond acceptors to achieve low Caco2 efflux for oral bioavailability of PARP inhibitors blocking centrosome clustering. Bioorg Med Chem Lett 26, 4775-4780 (2016).
21. I. T. Kirby, et al., A Potent and Selective PARP11 Inhibitor Suggests Coupling between Cellular Localization and Catalytic Activity. Cell chemical biology 25, 1547-1553.e12 (2018).
22. I. T. Kirby, R. K. Morgan, M. S. Cohen, “A Simple, Sensitive, and Generalizable Plate Assay for Screening PARP Inhibitors” in ADP-ribosylation and NAD+ Utilizing Enzymes., (2018), pp. 245-252.
23. D. I. James, et al., First-in-Class Chemical Probes against Poly(ADP-ribose) Glycohydrolase (PARG) Inhibit DNA Repair with Differential Pharmacology to Olaparib. Acs Chem Biol 11, 3179-3190 (2016).
24. J. Šileikytė, S. Sundalam, L. L. David, M. S. Cohen, Chemical Proteomics Approach for Profiling the NAD Interactome. J Am Chem Soc (2021) https:/doi.org/10.1021/jacs.1c01302.
25. E. Stewart, et al., Targeting the DNA repair pathway in Ewing sarcoma. Cell reports 9, 829-841 (2014).
26. S. J. Gill, et al., Combinations of PARP Inhibitors with Temozolomide Drive PARP1 Trapping and Apoptosis in Ewing's Sarcoma. Plos One 10, e0140988 (2015).
27. H. Hanzlikova, et al., The Importance of Poly(ADP-Ribose) Polymerase as a Sensor of Unligated Okazaki Fragments during DNA Replication. Molecular cell 71, 319-331.e3 (2018).
28. Y. Wang, R. Callejo, A. M. Z. Slawin, D. O'Hagan, The difluoromethylene (CF2) group in aliphatic chains: Synthesis and conformational preference of palmitic acids and nonadecane containing CF2 groups. Beilstein J Org Chem 10, 18-25 (2014).
29. C. Kim, X.-D. Wang, Y. Yu, PARP1 inhibitors trigger innate immunity via PARP1 trapping-induced DNA damage response. Elife 9, e60637 (2020).
30. M.-F. Langelier, J. L. Planck, K. M. Servent, J. M. Pascal, Poly(ADP-ribose) Polymerase, Methods and Protocols. Methods Mol Biology 780, 209-226 (2011).

What is claimed:

1. A compound of Formula (I): 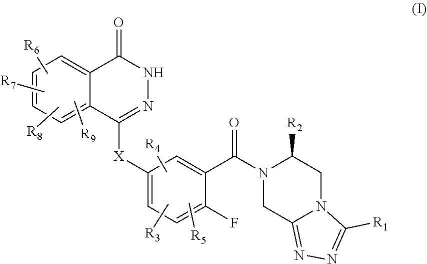
wherein:
X is selected from the group of —CH₂—, —CH(F)—, —C(F)₂—, —O—, —S—, —N(H)—, and —N(C₁-C₄ alkyl)-;
R₁ is selected from the group of C₁-C₄ alkyl, —O—C₁-C₄ alkyl, —S—C₁-C₄ alkyl, C₁-C₄ haloalkyl, —O—C₁-C₄ haloalkyl, —S—C₁-C₄ haloalkyl, C₃-C₆ cycloalkyl, —CH₂—C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl;
with each R₁ C₁-C₄ alkyl, —O—C₁-C₄ alkyl, —S—C₁-C₄ alkyl, C₃-C₆ cycloalkyl, —CH₂—C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl group being optionally substituted by 0, 1, 2, 3, 4, 5, or 6 substituents selected from OH, NH₂, NH(C₁-C₄ alkyl), N(C₁-C₄ alkyl)₂, and halogen; and
R₂ is C₁-C₄ alkyl, optionally substituted by halogen; or, when R₁ is —CH₂—CH₂—CH₃, or —CH₂—CH₂—CH₂—CH₃, R₂ may also be H;
R₃, R₄, R₅, R₆, R₇, and R₅ are each independently selected from the group of H, halogen, C₁-C₄ alkyl, —O—C₁-C₄ alkyl, phenyl, —O-phenyl, benzyl, —O-benzyl, —SO₂—C₁-C₄ alkyl, —SO₂—C₁-phenyl, —SO₂-benzyl, —NH₂, —NH(C₁-C₄ alkyl), —N(C₁-C₄ alkyl)₂, —NH-phenyl, and —NH-benzyl;
wherein the alkyl chains of the R₃, R₄, R₅, R₆, R₇, and R₈ C₁-C₄ alkyl, —O—C₁-C₄ alkyl, —SO₂—C₁-C₄ alkyl, NH(C₁-C₄ alkyl), and —N(C₁-C₄ alkyl)₂ groups may be optionally substituted by 0, 1, 2, 3, 4, or 5 substituents selected from Cl, F, CH₂F, CHF₂, CF₃, CF₂CF₃, OH, CN, NH₂, NH(C₁-C₄ alkyl), N(C₁-C₄ alkyl)₂, and NO₂;
and the rings of the R₃, R₄, R₅, R₆, R₇, and R₈ phenyl, —O-phenyl, benzyl, —O-benzyl, —SO₂—C₁-phenyl, —SO₂-benzyl, —NH-phenyl, and —NH-benzyl groups may be optionally substituted by 0, 1, 2, 3, 4, or 5 substituents selected from C₁-C₄ alkyl, Cl, F, CH₂F, CHF₂, CF₃, CF₂CF₃, OH, CN, NH₂, NH(C₁-C₄ alkyl), N(C₁-C₄ alkyl)₂, and NO₂;
R₉ is selected from the group of H, F, CH₂F, CHF₂, and CF₃;
with the proviso that no more than one of the group of R₃, R₄, and R₅ is phenyl, —O-phenyl, benzyl, —O-benzyl, —SO₂—C₁-phenyl, —SO₂-benzyl, —NH-phenyl, or —NH-benzyl;
with the proviso that no more than one of the group of R₇, R₈, and R₉ is phenyl, —O-phenyl, benzyl, —O-benzyl, —SO₂—C₁-phenyl, —SO₂-benzyl, —NH-phenyl, or —NH-benzyl; and
with the proviso that, when R₂ is CH₃ and each of R₃, R₄, R₅, R₆, R₇, R₈, and R₉ is H, then R₁ is not CF₂CF₃;
or a pharmaceutically acceptable salt, co-crystal, ester, solvate, hydrate, isomer (including optical isomers, racemates, or other mixtures thereof), tautomer, isotope, or polymorph thereof.

2. The compound of claim 1, or a pharmaceutically acceptable salt, co-crystal, ester, solvate, hydrate, isomer (including optical isomers, racemates, or other mixtures thereof), tautomer, isotope, or polymorph thereof, with the further proviso that, when R₂ is CH₃ and each of R₃, R₄, R₅, R₆, R₇, R₈, and R₉ is H, then R₁ is not CH₂F, CHF₂, CF₃, or CF₂CF₃;

3. The compound of any of claims 1 and 2, or a pharmaceutically acceptable salt, co-crystal, ester, solvate, hydrate, isomer (including optical isomers, racemates, or other mixtures thereof), tautomer, isotope, or polymorph thereof, wherein X is —CH₂—.

4. The compound of any of claims 1, 2, and 3, or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₄ alkyl, —O—C₁-C₄ alkyl, —S—C₁-C₄ alkyl, C₁-C₄ haloalkyl, —O—C₁-C₄ haloalkyl, and —S—C₁-C₄ haloalkyl.

5. The compound of any of claims 1, 2, 3, and 4, or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₃ alkyl, —O—C₁-C₃ alkyl, —S—C₁-C₃ alkyl, C₁-C₃ haloalkyl, —O—C₁-C₃ haloalkyl, and —S—C₁-C₃ haloalkyl; and all other variables (including R₂, R₃, R₄, R₅, R₆, R₇, R₈, and R₉) are as defined for Formula (I), above.

6. The compound of any of claims 1, 2, 3, 4, and 5, or a pharmaceutically acceptable salt, co-crystal, ester, solvate, hydrate, isomer (including optical isomers, racemates, or other mixtures thereof), tautomer, isotope, or polymorph thereof, wherein X is —CH₂—; and R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl.

7. The compound of any of claims 1, 2, 3, 4, 5, and 6, or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; and R₅ is H; R₆ is H.

8. The compound of any of claims 1, 2, 3, 4, 5, 6, and 7, or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₄ is H; R₅ is H; R₆ is H; R₇ is H; and all other variables (including R₂, R₃, R₈, and R₉) are as defined for Formula (I), above.

9. The compound of any of claims 1, 2, 3, 4, 5, 6, 7, and 8, or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₃ is H; R₄ is H; R₅ is H; R₆ is H; and R₇ is H.

10. A compound of Formula (II): 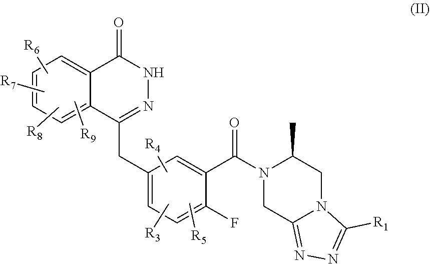
wherein:
R₁ is selected from the group of C₁-C₄ alkyl, —O—C₁-C₄ alkyl, —S—C₁-C₄ alkyl, C₁-C₄ haloalkyl, —O—C₁-C₄ haloalkyl, —S—C₁-C₄ haloalkyl, C₃-C₆ cycloalkyl, —CH₂—C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl;
with each R₁ C₁-C₄ alkyl, —O—C₁-C₄ alkyl, —S—C₁-C₄ alkyl, C₃-C₆ cycloalkyl, —CH₂—C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl group being optionally substituted by 0, 1, 2, 3, 4, 5, or 6 substituents selected from OH, NH₂, NH(C₁-C₄ alkyl), N(C₁-C₄ alkyl)₂, and halogen; and
R₃, R₄, R₅, R₆, R₇, and R₈ are each independently selected from the group of H, halogen, C₁-C₄ alkyl, —O—C₁-C₄ alkyl, phenyl, —O-phenyl, benzyl, —O-benzyl, —SO₂—C₁-C₄ alkyl, —SO₂—C₁-phenyl, —SO₂-benzyl, —NH₂, —NH(C₁-C₄ alkyl), —N(C₁-C₄ alkyl)₂, —NH-phenyl, and —NH-benzyl;
wherein the alkyl chains of the R₃, R₄, R₅, R₆, R₇, and R₈ C₁-C₄ alkyl, —O—C₁-C₄ alkyl, —SO₂—C₁-C₄ alkyl, NH(C₁-C₄ alkyl), and —N(C₁-C₄ alkyl)₂ groups may be optionally substituted by 0, 1, 2, 3, 4, or 5 substituents selected from Cl, F, CH₂F, CHF₂, CF₃, CF₂CF₃, OH, CN, NH₂, NH(C₁-C₄ alkyl), N(C₁-C₄ alkyl)₂, and NO₂;
and the rings of the R₃, R₄, R₅, R₆, R₇, and R₈ phenyl, —O-phenyl, benzyl, —O-benzyl, —SO₂—C₁-phenyl, —SO₂-benzyl, —NH-phenyl, and —NH-benzyl groups may be optionally substituted by 0, 1, 2, 3, 4, or 5 substituents selected from C₁-C₄ alkyl, Cl, F, CH₂F, CHF₂, CF₃, CF₂CF₃, OH, CN, NH₂, NH(C₁-C₄ alkyl), N(C₁-C₄ alkyl)₂, and NO₂;
R₉ is selected from the group of H, F, CH₂F, CHF₂, and CF₃;
with the proviso that no more than one of the group of R₃, R₄, and R₅ is phenyl, —O-phenyl, benzyl, —O-benzyl, —SO₂—C₁-phenyl, —SO₂-benzyl, —NH-phenyl, or —NH-benzyl;
with the proviso that no more than one of the group of R₇, R₈, and R₉ is phenyl, —O-phenyl, benzyl, —O-benzyl, —SO₂—C₁-phenyl, —SO₂-benzyl, —NH-phenyl, or —NH-benzyl; and
with the proviso that, when R₂ is CH₃ and each of R₃, R₄, R₅, R₆, R₇, R₈, and R₉ is H, then R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

11. The compound of claim 10, or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₄ alkyl, —O—C₁-C₄ alkyl, —S—C₁-C₄ alkyl, C₁-C₄ haloalkyl, —O—C₁-C₄ haloalkyl, and —S—C₁-C₄ haloalkyl.

12. The compound of any of claims 10 and 11, or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₃ alkyl, —O—C₁-C₃ alkyl, —S—C₁-C₃ alkyl, C₁-C₃ haloalkyl, —O—C₁-C₃ haloalkyl, and —S—C₁-C₃ haloalkyl; and all other variables (including R₃, R₄, R₅, R₆, R₇, R₈, and R₉) are as defined for Formula (II), above.

13. The compound of any of claims 10, 11, and 12, or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl.

14. The compound of any of claims 11, 12, and 13, or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₅ is H; and R₆ is H.

15. The compound of any of claims 11, 12, 13, and 14, or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₄ is H; R₅ is H; R₆ is H; and R₇ is H.

16. The compound of any of claims 11, 12, 13, 14, and 15, or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₃ is H; R₄ is H; R₅ is H; R₆ is H; R₇ is H; and all other variables (including R₈ and R₉) are as defined for Formula (II), above.

17. A compound of Formula (III): 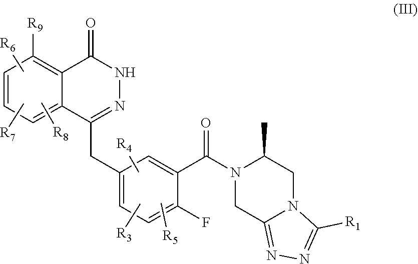
wherein:
R₁ is selected from the group of C₁-C₄ alkyl, —O—C₁-C₄ alkyl, —S—C₁-C₄ alkyl, C₁-C₄ haloalkyl, —O—C₁-C₄ haloalkyl, —S—C₁-C₄ haloalkyl, C₃-C₆ cycloalkyl, —CH₂—C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl;
with each R₁ C₁-C₄ alkyl, —O—C₁-C₄ alkyl, —S—C₁-C₄ alkyl, C₃-C₆ cycloalkyl, —CH₂—C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl group being optionally substituted by 0, 1, 2, 3, 4, 5, or 6 substituents selected from OH, NH₂, NH(C₁-C₄ alkyl), N(C₁-C₄ alkyl)₂, and halogen; and
R₃, R₄, R₅, R₆, R₇, and R₈ are each independently selected from the group of H, halogen, C₁-C₄ alkyl, —O—C₁-C₄ alkyl, phenyl, —O-phenyl, benzyl, —O-benzyl, —SO₂—C₁-C₄ alkyl, —SO₂—C₁-phenyl, —SO₂-benzyl, —NH₂, —NH(C₁-C₄ alkyl), —N(C₁-C₄ alkyl)₂, —NH-phenyl, and —NH-benzyl;
wherein the alkyl chains of the R₃, R₄, R₅, R₆, R₇, and R₈ C₁-C₄ alkyl, —O—C₁-C₄ alkyl, —SO₂—C₁-C₄ alkyl, NH(C₁-C₄ alkyl), and —N(C₁-C₄ alkyl)₂ groups may be optionally substituted by 0, 1, 2, 3, 4, or 5 substituents selected from Cl, F, CH₂F, CHF₂, CF₃, CF₂CF₃, OH, CN, NH₂, NH(C₁-C₄ alkyl), N(C₁-C₄ alkyl)₂, and NO₂;
and the rings of the R₃, R₄, R₅, R₆, R₇, and R₈ phenyl, —O-phenyl, benzyl, —O-benzyl, —SO₂—C₁-phenyl, —SO₂-benzyl, —NH-phenyl, and —NH-benzyl groups may be optionally substituted by 0, 1, 2, 3, 4, or 5 substituents selected from C₁-C₄ alkyl, Cl, F, CH₂F, CHF₂, CF₃, CF₂CF₃, OH, CN, NH₂, NH(C₁-C₄ alkyl), N(C₁-C₄ alkyl)₂, and NO₂;
R₉ is selected from the group of H, F, CH₂F, CHF₂, and CF₃;
with the proviso that no more than one of the group of R₃, R₄, and R₅ is phenyl, —O-phenyl, benzyl, —O-benzyl, —SO₂—C₁-phenyl, —SO₂-benzyl, —NH-phenyl, or —NH-benzyl;
with the proviso that no more than one of the group of R₇, R₈, and R₉ is phenyl, —O-phenyl, benzyl, —O-benzyl, —SO₂—C₁-phenyl, —SO₂-benzyl, —NH-phenyl, or —NH-benzyl; and
with the proviso that, when R₂ is CH₃ and each of R₃, R₄, R₅, R₆, R₇, R₈, and R₉ is H, then R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

18. The compound of claim 17, or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₄ alkyl, —O—C₁-C₄ alkyl, —S—C₁-C₄ alkyl, C₁-C₄ haloalkyl, —O—C₁-C₄ haloalkyl, and —S—C₁-C₄ haloalkyl.

19. A compound of any of claims 17 and 18, or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₃ alkyl, —O—C₁-C₃ alkyl, —S—C₁-C₃ alkyl, C₁-C₃ haloalkyl, —O—C₁-C₃ haloalkyl, and —S—C₁-C₃ haloalkyl.

20. A compound of any of claims 17, 18, and 19, or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl.

21. A compound of any of claims 17, 18, 19, and 20, or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₅ is H; R₆ is H.

22. A compound of any of claims 17, 18, 19, 20, and 21, or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₄ is H; R₅ is H; R₆ is H; R₇ is H.

23. A compound of any of claims 17, 18, 19, 20, 21, and 22, or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₃ is H; R₄ is H; R₅ is H; R₆ is H; R₇ is H.

24. A compound of Formula (IV): 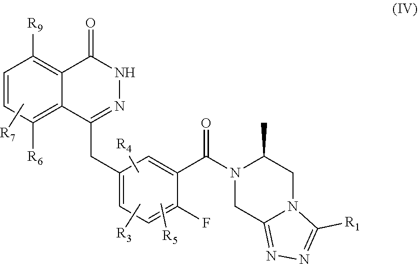
wherein:
R₁ is selected from the group of C₁-C₄ alkyl, —O—C₁-C₄ alkyl, —S—C₁-C₄ alkyl, C₁-C₄ haloalkyl, —O—C₁-C₄ haloalkyl, —S—C₁-C₄ haloalkyl, C₃-C₆ cycloalkyl, —CH₂—C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl;
with each R₁ C₁-C₄ alkyl, —O—C₁-C₄ alkyl, —S—C₁-C₄ alkyl, C₃-C₆ cycloalkyl, —CH₂—C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl group being optionally substituted by 0, 1, 2, 3, 4, 5, or 6 substituents selected from OH, NH₂, NH(C₁-C₄ alkyl), N(C₁-C₄ alkyl)₂, and halogen; and
R₃, R₄, R₅, R₆, R₇, and R₅ are each independently selected from the group of H, halogen, C₁-C₄ alkyl, —O—C₁-C₄ alkyl, phenyl, —O-phenyl, benzyl, —O-benzyl, —SO₂—C₁-C₄ alkyl, —SO₂—C₁-phenyl, —SO₂-benzyl, —NH₂, —NH(C₁-C₄ alkyl), —N(C₁-C₄ alkyl)₂, —NH-phenyl, and —NH-benzyl;
wherein the alkyl chains of the R₃, R₄, R₅, R₆, R₇, and R₈ C₁-C₄ alkyl, —O—C₁-C₄ alkyl, —SO₂—C₁-C₄ alkyl, NH(C₁-C₄ alkyl), and —N(C₁-C₄ alkyl)₂ groups may be optionally substituted by 0, 1, 2, 3, 4, or 5 substituents selected from Cl, F, CH₂F, CHF₂, CF₃, CF₂CF₃, OH, CN, NH₂, NH(C₁-C₄ alkyl), N(C₁-C₄ alkyl)₂, and NO₂;
and the rings of the R₃, R₄, R₅, R₆, R₇, and R₈ phenyl, —O-phenyl, benzyl, —O-benzyl, —SO₂—C₁-phenyl, —SO₂-benzyl, —NH-phenyl, and —NH-benzyl groups may be optionally substituted by 0, 1, 2, 3, 4, or 5 substituents selected from C₁-C₄ alkyl, Cl, F, CH₂F, CHF₂, CF₃, CF₂CF₃, OH, CN, NH₂, NH(C₁-C₄ alkyl), N(C₁-C₄ alkyl)₂, and NO₂;
R₉ is selected from the group of H, F, CH₂F, CHF₂, and CF₃;
with the proviso that no more than one of the group of R₃, R₄, and R₅ is phenyl, —O-phenyl, benzyl, —O-benzyl, —SO₂—C₁-phenyl, —SO₂-benzyl, —NH-phenyl, or —NH-benzyl;
with the proviso that no more than one of the group of R₇, R₈, and R₉ is phenyl, —O-phenyl, benzyl, —O-benzyl, —SO₂—C₁-phenyl, —SO₂-benzyl, —NH-phenyl, or —NH-benzyl; and
with the proviso that, when R₂ is CH₃ and each of R₃, R₄, R₅, R₆, R₇, R₈, and R₉ is H, then R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

25. The compound of claim 24, or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₄ alkyl, —O—C₁-C₄ alkyl, —S—C₁-C₄ alkyl, C₁-C₄ haloalkyl, —O—C₁-C₄ haloalkyl, and —S—C₁-C₄ haloalkyl.

26. The compound of any of claims 24 and 25, or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₃ alkyl, —O—C₁-C₃ alkyl, —S—C₁-C₃ alkyl, C₁-C₃ haloalkyl, —O—C₁-C₃ haloalkyl, and —S—C₁-C₃ haloalkyl.

27. The compound of any of claims 24, 25, and 26, or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl.

28. The compound of any of claims 24, 25, 26, and 27, or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₆ is H; R₆ is H.

29. The compound of any of claims 24, 25, 26, 27, and 28, or a pharmaceutically acceptable salt, co-crystal, ester, solvate, hydrate, isomer (including optical isomers, racemates, or other mixtures thereof), tautomer, isotope, or polymorph thereof, wherein R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₄ is H; R₅ is H; R₆ is H; R₇ is H; and all other variables (including R₃, and R₉) are as defined for Formula (IV), above.

30. The compound of any of claims 24, 25, 26, 27, 28, and 29, or a pharmaceutically acceptable salt, co-crystal, ester, solvate, hydrate, isomer (including optical isomers, racemates, or other mixtures thereof), tautomer, isotope, or polymorph thereof, wherein X is —CH₂—; R₁ is selected from the group of C₁-C₂ alkyl, —O—C₁-C₂ alkyl, —S—C₁-C₂ alkyl, C₁-C₂ haloalkyl, —O—C₁-C₂ haloalkyl, and —S—C₁-C₂ haloalkyl; R₃ is H; R₄ is H; R₅ is H; R₆ is H; R₇ is H.

31. A compound of Formula (V): 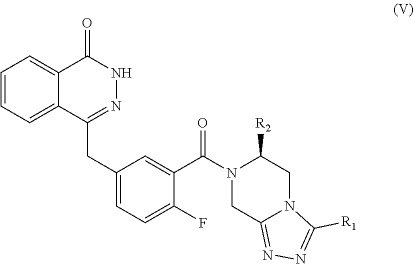
wherein:
R₁ is selected from the group of C₁-C₄ alkyl, —O—C₁-C₄ alkyl, —S—C₁-C₄ alkyl, C₁-C₄ haloalkyl, —O—C₁-C₄ haloalkyl, —S—C₁-C₄ haloalkyl, C₃-C₆ cycloalkyl, —CH₂—C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl;
with each R₁ C₁-C₄ alkyl, —O—C₁-C₄ alkyl, —S—C₁-C₄ alkyl, C₃-C₆ cycloalkyl, —CH₂—C₃-C₆ cycloalkyl, —O—C₃-C₆ cycloalkyl, 5-membered heterocyclyl, —O-5-membered heterocyclyl, 6-membered heterocyclyl, and —O-6-membered heterocyclyl group being optionally substituted by 0, 1, 2, 3, 4, 5, or 6 substituents selected from OH, NH₂, NH(C₁-C₄ alkyl), N(C₁-C₄ alkyl)₂, and halogen;
R₂ is C₁-C₄ alkyl; or, when R₁ is —CH₂—CH₂—CH₃, or —CH₂—CH₂—CH₂—CH₃, R₂ may also be H;
with the proviso that, when R₂ is CH₃, R₁ is not CF₂CF₃;
or a pharmaceutically acceptable salt, co-crystal, ester, solvate, hydrate, isomer (including optical isomers, racemates, or other mixtures thereof), tautomer, isotope, or polymorph thereof.

32. The compound of claim 31, wherein:
R₁ is selected from the group of C₁-C₄ haloalkyl, C₃-C₄ cycloalkyl, —CH₂—C₃-C₄ cycloalkyl, and C₁-C₃ alkyl, wherein the R₁ C₃-C₄ cycloalkyl, —CH₂—C₃-C₄ cycloalkyl, and C₁-C₃ alkyl groups are optionally substituted by substituents selected from OH, NH₂, and halogen; and
R₂ is C₁-C₄ alkyl; or, when R₁ is —CH₂—CH₂—CH₃, R₂ may also be H;
with the proviso that, when R₂ is CH₃, R₁ is not CF₂CF₃;
or a pharmaceutically acceptable salt, co-crystal, ester, solvate, hydrate, isomer (including optical isomers, racemates, or other mixtures thereof), tautomer, isotope, or polymorph thereof.

33. The compound of any of claims 31 and 32, wherein:
R₁ is selected from the group of C₁-C₃ haloalkyl, C₃-C₄ cycloalkyl, —CH₂—C₃-C₄ cycloalkyl, and C₁-C₃ alkyl, wherein the R₁ C₃-C₄ cycloalkyl, —CH₂—C₃-C₄ cycloalkyl, and C₁-C₃ alkyl groups are optionally substituted by substituents selected from OH, NH₂, and halogen; and
R₂ is C₁-C₄ alkyl; or, when R₁ is —CH₂—CH₂—CH₃, R₂ may also be H;
with the proviso that, when R₂ is CH₃, R₁ is not CF₂CF₃;
or a pharmaceutically acceptable salt, co-crystal, ester, solvate, hydrate, isomer (including optical isomers, racemates, or other mixtures thereof), tautomer, isotope, or polymorph thereof.

34. A compound of Formula (VI): 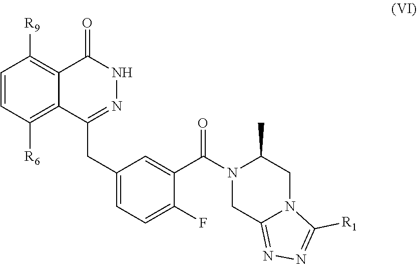
wherein,
R₁ is C₁-C₄ haloalkyl;
R₆ is selected from the group of H, halogen, C₁-C₄ alkyl, —O—C₁-C₄ alkyl, phenyl, —O-phenyl, benzyl, —O-benzyl, —SO₂—C₁-C₄ alkyl, —SO₂—C₁-phenyl, —SO₂-benzyl, —NH₂, —NH(C₁-C₄ alkyl), —N(C₁-C₄ alkyl)₂, —NH-phenyl, and —NH-benzyl;
wherein the alkyl chains of the R₆ C₁-C₄ alkyl, —O—C₁-C₄ alkyl, —SO₂—C₁-C₄ alkyl, NH(C₁-C₄ alkyl), and —N(C₁-C₄ alkyl)₂ groups may be optionally substituted by 0, 1, 2, 3, 4, or 5 substituents selected from Cl, F, CH₂F, CHF₂, CF₃, CF₂CF₃, OH, CN, NH₂, NH(C₁-C₄ alkyl), N(C₁-C₄ alkyl)₂, and NO₂;
and the rings of the R₆ phenyl, —O-phenyl, benzyl, —O-benzyl, —SO₂—C₁-phenyl, —SO₂-benzyl, —NH-phenyl, and —NH-benzyl groups may be optionally substituted by 0, 1, 2, 3, 4, or 5 substituents selected from C₁-C₄ alkyl, Cl, F, CH₂F, CHF₂, CF₃, CF₂CF₃, OH, CN, NH₂, NH(C₁-C₄ alkyl), N(C₁-C₄ alkyl)₂, and NO₂;
R₉ is selected from the group of H, F, CH₂F, CHF₂, and CF₃;
with the proviso that, when R₆ is H and Ry is H, then R₁ is not CF₂CF₃;
or a pharmaceutically acceptable salt, co-crystal, ester, solvate, hydrate, isomer (including optical isomers, racemates, or other mixtures thereof), tautomer, isotope, or polymorph thereof.

35. The compound of claim 34, wherein:
R₁ is C₁-C₄ haloalkyl;
R₆ is selected from the group of H, halogen, C₁-C₄ alkyl, —O—C₁-C₄ alkyl, —NH₂, —NH(C₁-C₄ alkyl), and —N(C₁-C₄ alkyl)₂;
wherein the alkyl chains of the R₆ C₁-C₄ alkyl, —O—C₁-C₄ alkyl, NH(C₁-C₄ alkyl), and —N(C₁-C₄ alkyl)₂ groups may be optionally substituted by 0, 1, 2, 3, 4, or 5 substituents selected from Cl, F, CH₂F, CHF₂, CF₃, CF₂CF₃, OH, CN, NH₂, NH(C₁-C₄ alkyl), N(C₁-C₄ alkyl)₂, and NO₂;
R₉ is selected from the group of H, F, CH₂F, CHF₂, and CF₃;
with the proviso that, when R₆ is H and Ry is H, then R₁ is not CF₂CF₃;
or a pharmaceutically acceptable salt, co-crystal, ester, solvate, hydrate, isomer (including optical isomers, racemates, or other mixtures thereof), tautomer, isotope, or polymorph thereof.

36. The compound of any of claims 34 and 35, wherein:
R₁ is C₁-C₃ haloalkyl;
R₆ is selected from the group of H, halogen, C₁-C₄ alkyl, —O—C₁-C₄ alkyl, —NH₂, —NH(C₁-C₄ alkyl), and —N(C₁-C₄ alkyl)₂;
wherein the alkyl chains of the R₆ C₁-C₄ alkyl, —O—C₁-C₄ alkyl, NH(C₁-C₄ alkyl), and —N(C₁-C₄ alkyl)₂ groups may be optionally substituted by 0, 1, 2, 3, 4, or 5 substituents selected from Cl, F, CH₂F, CHF₂, CF₃, CF₂CF₃, OH, CN, NH₂, NH(C₁-C₄ alkyl), N(C₁-C₄ alkyl)₂, and NO₂;
R₉ is selected from the group of H, F, CH₂F, CHF₂, and CF₃;
with the proviso that, when R₆ is H and R₉ is H, then R₁ is not CF₂CF₃;
or a pharmaceutically acceptable salt, co-crystal, ester, solvate, hydrate, isomer (including optical isomers, racemates, or other mixtures thereof), tautomer, isotope, or polymorph thereof.

37. A compound of Formula (VII): 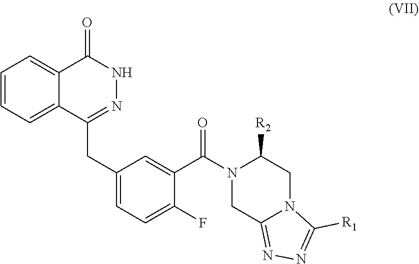
wherein:
R₁ is selected from the group of C₃-C₈ cycloalkyl, —CH₂—C₃-C₈ cycloalkyl, and C₁-C₄ alkyl optionally substituted by substituents selected from OH, NH₂, and halogen; and
R₂ is C₁-C₄ alkyl; or, when R₁ is —CH₂—CH₂—CH₃, or —CH₂—CH₂—CH₂—CH₃, R₂ may also be H;
with the proviso that, when R₂ is CH₃, R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

38. The compound of claim 37, wherein:
R₁ is selected from the group of C₃-C₄ cycloalkyl, —CH₂—C₃-C₄ cycloalkyl, and C₁-C₃ alkyl optionally substituted by substituents selected from OH, NH₂, and halogen; and
R₂ is C₁-C₄ alkyl; or, when R₁ is —CH₂—CH₂—CH₃, R₂ may also be H;
with the proviso that, when R₂ is CH₃, R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

39. The compound of any of claims 37 and 38, wherein:
R₁ is C₁-C₃ alkyl optionally substituted by substituents selected from OH, NH₂, and halogen; and
R₂ is C₁-C₄ alkyl; or, when R₁ is —CH₂—CH₂—CH₃, R₂ may also be H;
with the proviso that, when R₂ is CH₃, R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

40. The compound of any of claims 37, 38, and 39, wherein:
R₁ is C₁-C₃ alkyl optionally substituted by substituents selected from OH, NH₂, and F; and
R₂ is C₁-C₄ alkyl; or, when R₁ is —CH₂—CH₂—CH₃, R₂ may also be H;
with the proviso that, when R₂ is CH₃, R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

41. The compound of any of claims 37, 38, 39, and 40, wherein:
R₁ is C₁-C₃ alkyl optionally substituted by one or more F substituents; and
R₂ is C₁-C₃ alkyl; or, when R₁ is —CH₂—CH₂—CH₃, R₂ may also be H;
with the proviso that, when R₂ is CH₃, R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

42. The compound of any of claims 37, 38, 39, 41, and 42, wherein:
R₁ is C₁-C₃ alkyl optionally substituted by one or more F substituents; and
R₂ is CH₃;
with the proviso that, when R₂ is CH₃, R₁ is not CH₂F, CHF₂, CF₃, or CF₂CF₃;
or a pharmaceutically acceptable salt, co-crystal, ester, solvate, hydrate, isomer (including optical isomers, racemates, or other mixtures thereof), tautomer, isotope, or polymorph thereof.

43. The compound of any of claims 37, 38, 39, 40, 41, and 42, wherein:
R₁ is selected from the group of —CHF—CH₃, —CF₂—CH₃, —CH₂—CH₂F, —CH₂—CHF₂, —CH₂—CF₃, and —CHF—CF₃; and
R₂ is CH₃;
or a pharmaceutically acceptable salt, co-crystal, ester, solvate, hydrate, isomer (including optical isomers, racemates, or other mixtures thereof), tautomer, isotope, or polymorph thereof.

44. The compound of any of claims 37, 38, 39, 40, 41, 42, and 43, wherein:
R₁ is selected from the group of —CHF—CH₃ and —CF₂—CH₃; and
R₂ is CH₃;
or a pharmaceutically acceptable salt, co-crystal, ester, solvate, hydrate, isomer (including optical isomers, racemates, or other mixtures thereof), tautomer, isotope, or polymorph thereof.

45. A compound having the structure: 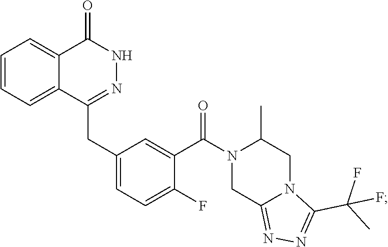 [SMILES: CC1Cn2c(nnc2C(C)(F)F)CN1C(=O)c1cc(Cc2n[nH]c(=O)c3ccccc23)ccc1F]
or a pharmaceutically acceptable salt, co-crystal, ester, solvate, hydrate, isomer (including optical isomers, racemates, or other mixtures thereof), tautomer, isotope, or polymorph thereof.

46. The compound: 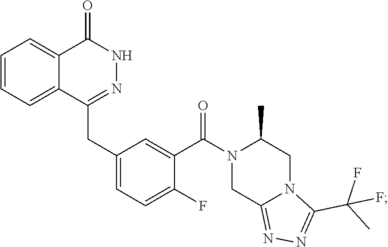 [SMILES: C[C@H]1Cn2c(nnc2C(C)(F)F)CN1C(=O)c1cc(Cc2n[nH]c(=O)c3ccccc23)ccc1F]
or a pharmaceutically acceptable salt, co-crystal, ester, solvate, hydrate, isomer (including optical isomers, racemates, or other mixtures thereof), tautomer, isotope, or polymorph thereof.

47. The compound: 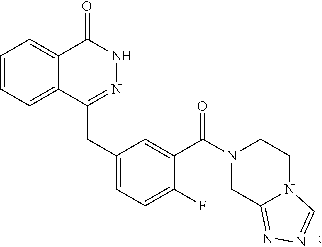 [SMILES: O=C(c1cc(Cc2n[nH]c(=O)c3ccccc23)ccc1F)N1CCn2cnnc2C1]
or a pharmaceutically acceptable salt, co-crystal, ester, solvate, hydrate, isomer (including optical isomers, racemates, or other mixtures thereof), tautomer, isotope, or polymorph thereof.

48. The compound: 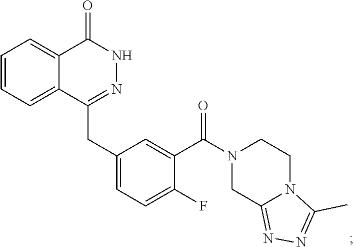 [SMILES: Cc1nnc2n1CCN(C(=O)c1cc(Cc3n[nH]c(=O)c4ccccc34)ccc1F)C2]
or a pharmaceutically acceptable salt, co-crystal, ester, solvate, hydrate, isomer (including optical isomers, racemates, or other mixtures thereof), tautomer, isotope, or polymorph thereof.

49. The compound: 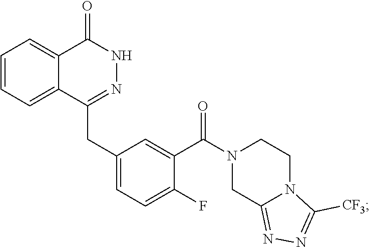 [SMILES: O=C(c1cc(Cc2n[nH]c(=O)c3ccccc23)ccc1F)N1CCn2c(nnc2C(F)(F)F)C1]
or a pharmaceutically acceptable salt, co-crystal, ester, solvate, hydrate, isomer (including optical isomers, racemates, or other mixtures thereof), tautomer, isotope, or polymorph thereof.

50. The compound: 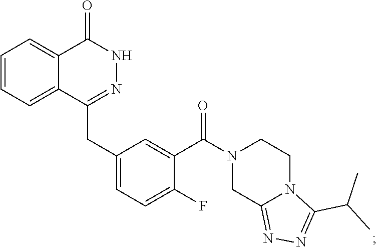 [SMILES: CC(C)c1nnc2n1CCN(C(=O)c1cc(Cc3n[nH]c(=O)c4ccccc34)ccc1F)C2]
or a pharmaceutically acceptable salt, co-crystal, ester, solvate, hydrate, isomer (including optical isomers, racemates, or other mixtures thereof), tautomer, isotope, or polymorph thereof.

51. A pharmaceutical composition comprising a therapeutically effective amount of a compound selected from any of claims 1 through 50, or a pharmaceutically acceptable salt, co-crystal, ester, solvate, hydrate, isomer (including optical isomers, racemates, or other mixtures thereof), tautomer, isotope, or polymorph thereof, and a pharmaceutically acceptable carrier or excipient.

52. The pharmaceutical composition of claim 51, wherein the therapeutically effective amount comprises a single dose of from about 1 mg to about 1,000 mg.

53. The pharmaceutical composition of claim 51, wherein the therapeutically effective amount comprises a single dose of from about 50 mg to about 500 mg.

54. The use of a compound selected from any of claims 1 through 50, or a pharmaceutically acceptable salt, co-crystal, ester, solvate, hydrate, isomer (including optical isomers, racemates, or other mixtures thereof), tautomer, isotope, or polymorph thereof, in the preparation of a medicament.

55. A method of treatment in a subject of a disease or condition characterized by overexpression of PARP-1, the method comprising administering to the subject in need thereof a therapeutically effective amount of a compound selected from any of claims 1 through 50, or a pharmaceutically acceptable salt, co-crystal, ester, solvate, hydrate, isomer (including optical isomers, racemates, or other mixtures thereof), tautomer, isotope, or polymorph thereof, in the preparation of a medicament.

56. The method of claim 55, wherein the disease or condition characterized by overexpression of PARP-1 is selected from the group of ovarian cancer, deleterious BRCA mutation (germline and/or somatic)-associated epithelial ovarian cancer, fallopian tube cancer, cervical cancer (including recurrent cervical cancer), or primary peritoneal cancer (including those in a complete or partial response to platinum-based chemotherapy, as well as those in subjects who have been treated with two or more chemotherapies), stomach cancer, colorectal cancer, prostate cancer including metastatic castration-resistant prostate cancer), lymphomas, BRCA mutant lung cancer, melanomas, breast cancer (including triple negative breast cancer and HER2+ breast cancer), lung cancer, Ewing sarcoma, osteosarcoma, glioblastoma, lymphoma, skin cancer, kidney cancer, testicular cancer, and leukemia.

57. The method of claim 55, wherein the disease or condition characterized by overexpression of PARP-1 is a neurodegenerative disorder.

58. The method of claim 57, wherein the neurodegenerative disorder is selected from the group of Alzheimer's disease and Parkinson's disease.

59. The method of claim 55, wherein the disease or condition characterized by overexpression of PARP-1 is breast cancer.

60. The method of any of claims 55 and 59, wherein the disease or condition characterized by overexpression of PARP-1 is triple negative breast cancer.

61. The method of any of claims 55 and 59, wherein the disease or condition characterized by overexpression of PARP-1 is HER2+ breast cancer.

62. The method of claim 55, wherein the disease or condition characterized by overexpression of PARP-1 is Ewing sarcoma.

63. The method of claim 55, wherein the disease or condition characterized by overexpression of PARP-1 is ovarian cancer.

64. The method of any of claims 55 and 63, wherein the disease or condition characterized by overexpression of PARP-1 is deleterious BRCA mutation (germline and/or somatic)-associated epithelial ovarian cancer.

65. The method of claim 55, wherein the disease or condition characterized by overexpression of PARP-1 is prostate cancer.

66. The method of any of claims 55 and 65, wherein the disease or condition characterized by overexpression of PARP-1 is metastatic castration-resistant prostate cancer.

67. The method of claim 55, wherein the disease or condition characterized by overexpression of PARP-1 is pancreatic cancer.

68. The method of any of claims 55 and 67, wherein the disease or condition characterized by overexpression of PARP-1 is pancreatic ductal adenocarcinoma.

69. The method of claim 55, wherein the disease or condition characterized by overexpression of PARP-1 is fallopian tube cancer.

70. The method of claim 55, wherein the disease or condition characterized by overexpression of PARP-1 is cervical cancer.

71. The method of any of claims 55 and 70, wherein the disease or condition characterized by overexpression of PARP-1 is recurrent cervical cancer.

72. The method of claim 55, wherein the disease or condition characterized by overexpression of PARP-1 is primary peritoneal cancer.

73. The method of claim 55, wherein the disease or condition characterized by overexpression of PARP-1 is stomach cancer.

74. The method of claim 55, wherein the disease or condition characterized by overexpression of PARP-1 is a lymphoma.

75. The method of claim 55, wherein the disease or condition characterized by overexpression of PARP-1 is a melanoma.

76. The method of claim 55, wherein the disease or condition characterized by overexpression of PARP-1 is lung cancer.

77. The method of any of claims 55 and 75, wherein the disease or condition characterized by overexpression of PARP-1 is BRCA mutant lung cancer.

78. The method of claim 55, wherein the disease or condition characterized by overexpression of PARP-1 is an osteosarcoma.

79. The method of claim 55, wherein the disease or condition characterized by overexpression of PARP-1 is a glioblastoma.

80. The method of claim 55, wherein the disease or condition characterized by overexpression of PARP-1 is colorectal cancer.

81. The method of claim 55, wherein the disease or condition characterized by overexpression of PARP-1 is skin cancer.

82. The method of claim 55, wherein the disease or condition characterized by overexpression of PARP-1 is kidney cancer.

83. The method of claim 55, wherein the disease or condition characterized by overexpression of PARP-1 is testicular cancer.

81. The method of claim 55, wherein the disease or condition characterized by overexpression of PARP-1 is a leukemia.


SEQUENCE LISTING (6 sequences)

SEQ ID NO: 1 - DNA, 26 bp, synthetic construct
  source 1..26: mol_type = other DNA
gactcagctagcgggccagttctgga

SEQ ID NO: 2 - DNA, 26 bp, synthetic construct
  source 1..26: mol_type = other DNA
tgagtcgaattctctgagttccgatc

SEQ ID NO: 3 - DNA, 25 bp, synthetic construct
  source 1..25: mol_type = other DNA
agctaggctgagcaaaaggcagatc

SEQ ID NO: 4 - DNA, 26 bp, synthetic construct
  source 1..26: mol_type = other DNA
ctagctgatatcgacttccaagtcat

SEQ ID NO: 5 - DNA, 26 bp, synthetic construct
  source 1..26: mol_type = other DNA
agctaggatatctcgtgaaggcgaat

SEQ ID NO: 6 - DNA, 23 bp, synthetic construct
  source 1..23: mol_type = other DNA
ctagctaccggtggatcccggga